(19) United States
(12) Patent Application Publication
(10) Pub. No.: US 2017/0087344 A1
(43) Pub. Date: Mar. 30, 2017

(54) DEVICES AND METHODS FOR REDUCING THE RISK OF PRETERM LABOR AND PRETERM BIRTH

(71) Applicant: THERAPEUTIC SOLUTIONS INTERNATIONAL, INC., Oceanside, CA (US)

(72) Inventor: Thomas Ichim, San Diego, CA (US)

(21) Appl. No.: 15/274,080

(22) Filed: Sep. 23, 2016

Related U.S. Application Data
(60) Provisional application No. 62/232,722, filed on Sep. 25, 2015.

(51) Int. Cl. A61M 31/00; A61L 31/16; A61B 17/42; A61M 27/00; A61M 39/24; A61L 31/14; A61L 31/06

(52) U.S. Cl. CPC A61M 31/002; A61L 31/148; A61L 31/16; A61L 31/06; A61M 27/008; A61M 39/24; A61B 17/4241; A61M 2210/1433

(57) ABSTRACT

Devices for reducing the risk of pregnancy complications, including preterm labor and preterm birth, are disclosed herein. Also disclosed are devices in combination with therapies and medicaments for the treatment of pregnancy complications. Also disclosed are methods of using the devices for the treatment of pregnancy complications.

### CROSS-REFERENCE TO RELATED APPLICATIONS

The present application claims the benefit of priority to U.S. Provisional Patent Application No. 62/232,722, filed Sep. 25, 2015, the disclosure of which is incorporated by reference herein in its entirety.

### FIELD

The present disclosure generally relates to the field of treating diseases, disorders, or conditions in a subject mammal. In particular, the present disclosure relates generally to medical devices, and methods of use for these devices, which are used to reduce the risk of preterm labor and preterm birth. The present disclosure also generally relates to the field of delivering therapeutically effective amounts of medicaments, facilitated by the medical devices described herein, for use in treating diseases, disorders, or conditions in a subject mammal.

### BACKGROUND

Pregnancy complications impose tremendous costs on the U.S. healthcare system, and worldwide. Of all pregnancy complications, preterm birth is the most prevalent. Preterm birth (PTB) is defined as delivering after 22 weeks, and before 37 weeks (Hanna, Nazeeh, and Daniel Kiefer. “A translational view of biomarkers in preterm labor.” American Journal of Reproductive Immunology 67.4 (2012): 268-272; Murtha, Amy P., et al. “Maternal serum interleukin-6 concentrations in patients with preterm premature rupture of membranes and evidence of infection.” American Journal of Obstetrics and Gynecology 175.4 (1996): 966-969; Institute of Medicine of the National Academies. Preterm Birth: Causes, Consequences, and Prevention. Wash. D.C.: The National Academies Press, 2007). Premature deliveries account for about 12.5% of all births in the U.S., and that number is even higher for African American women (17.8%) (Institute of Medicine of the National Academies, Preterm Birth: Causes, Consequences, and Prevention. Wash. D.C.: The National Academies Press, 2007). In addition to the high financial costs, pregnancy complications such as spontaneous preterm labor and preterm births are the most prevalent reasons for perinatal morbidity and mortality, cerebral palsy, other mental and cognitive developmental issues, blindness, deafness, and other disorders (Hanna, Nazeeh, and Daniel Kiefer. “A translational view of biomarkers in preterm labor.” American Journal of Reproductive Immunology 67.4 (2012): 268-272). In 2005, the annual cost of premature babies was more than 26 billion dollars (Murtha, Amy P., et al. “Maternal serum interleukin-6 concentrations in patients with preterm premature rupture of membranes and evidence of infection,” American Journal of Obstetrics and Gynecology 175.4 (1996): 966-969). In addition, preterm premature rupture of the membranes (PPROM) accounts for 2% of all pregnancies, and is associated with 20% of all perinatal deaths (Murtha, Amy P., et al. “Maternal serum interleukin-6 concentrations in patients with preterm premature rupture of membranes and evidence of infection.” American Journal of Obstetrics and Gynecology 175.4 (1996): 966-969). The incidence of premature births, and resulting health implications, have been on the rise, and to date much research has been performed, but no substantial improvements made in the field (Hanna, Nazeeh, and Daniel Kiefer. “A translational view of biomarkers in preterm labor.” American Journal of Reproductive Immunology 67.4 (2012): 268-272; Gonzalez, Juan M., et al, “Complement activation triggers metalloproteinases release inducing cervical remodeling and preterm birth in mice.” The American Journal of Pathology 179.2 (2011): 838-849). The citations referred to herein are incorporated by reference in their entireties.

Preterm birth is a multifactorial syndrome, with a heterogeneous etiology. Triggers leading to preterm birth can involve “behavioral and psychosocial factors, socioeconomic status, environmental exposures, medical conditions, a history of infertility treatments, biological factors, and genetics,” as well as over-vigorous sexual intercourse or extreme physical exercise, bacterial vaginosis (vaginal biome dysbiosis), poor oral care (periodontitis or gingivitis) leading to bacterial infection of the maternal peripheral blood compartment, or some combination of any and all of these factors (Institute of Medicine of the National Academies. Preterm Birth: Causes, Consequences, and Prevention. Wash. D.C.: The National Academies Press, 2007). There is little known about the mechanisms involved that lead to preterm birth (Gonzalez, Juan M., et al. “Complement activation triggers metalloproteinases release inducing cervical remodeling and preterm birth in mice.” The American Journal of Pathology 179.2 (2011): 838-849). Heredity is thought to be an important factor in predicting PTB, where previous PTB in the woman or in her family, is taken as a predictor (Ehn, Nicole L., et al. “Evaluation of fetal and maternal genetic variation in the progesterone receptor gene for contributions to preterm birth.” Pediatric Research 62.5 (2007): 630-635). A twin study revealed that PTB has a heritability of around 40% (Ehn, Nicole L., et al. “Evaluation of fetal and maternal genetic variation in the progesterone receptor gene for contributions to preterm birth,” Pediatric Research 62.5 (2007): 630-635). This is most likely the reason it has been difficult to define a diagnostic fingerprint for predicting the likelihood of a female mammal to deliver preterm. What is believed, however, is that both normal birth and even preterm birth, is an inflammatory process. This complex process may involve activation and interactions of various proinflammatory mediators and inflammatory cytokines, and various classes of prostaglandins, proteins or enzymes such as matrix metalloproteinases that alter the integrity of the cervix and secondarily the integrity of the cervical mucus plug, thus allowing bacterial infection of the amnion, chorion, and placental compartment, causing the membranes to rupture, the cervix to relax, and the uterus to contract, leading to preterm birth (Vadillo-Ortega. Felipe, and Guadalupe Estrada-Gutiérrez. “Role of matrix metalloproteinases in preterm labour.” BJOG: An International Journal of Obstetrics & Gynecology 112.s1 (2005): 19-22). The citations referred to herein are incorporated by reference in their entireties.

Thus, preservation of the integrity of the cervical mucus plug is essential to preventing preterm labor and ensuring full-term births. This need is especially critical for mammals in need with shorter than average cervixes and therefore shorter mucus plugs.

Interestingly, 30% of spontaneous preterm labor is believed to be due to preterm premature rupture of the membranes (PPROM), which is the highest percentage of any factor known to he associated with PTB (Vadillo-Ortega, Felipe, and Guadalupe Estrada-Gutiérrez. “Role of matrix metalloproteinases in preterm labour.” BJOG.. An International Journal of Obstetrics & Gynecology 112.s1 (2005): 19-22). PROM is defined as “the transvaginal loss of amniotic fluid in the absence of active labor at any gestational age,” and it is especially important from a clinical standpoint if it occurs prematurely (PPROM), or prior to 37 weeks gestation (Vadillo-Ortega, Felipe, and Guadalupe Estrada-Gutiérrez. “Role of matrix metalloproteinases in preterm labour.” BJOG: An International Journal of Obstetrics & Gynecology 112.s1 (2005): 19-22; incorporated by reference herein in its entirety).

Gestational diabetes is another significant problem that can occur during pregnancy, and which can cause health problems for both the mother and the baby. Gestational diabetes affects about 18% of all pregnancies, and it arises in women during pregnancy due to high blood glucose levels, even if there is no prior history of diabetes. As with PTB and PROM/PPROM, the causes of gestational diabetes are not fully understood. One proposed explanation for this condition is linked to the placental hormones that are essential for the baby's development. While these hormones are beneficial to the fetal development, they block the action of insulin in the mother.

The failure of insulin receptors to function properly results in a buildup of insulin and glucose in the blood stream. This leads to the inability of the body to use the required amount of insulin needed for the pregnancy, signifying the onset of gestational diabetes. If this condition is not properly treated it can have negative effects on the baby. The high levels of maternal blood glucose will leak into the placenta, leading to high blood glucose levels in the baby, resulting in elevated levels of insulin production by the baby's pancreas. Elevated blood glucose levels in the baby are dangerous, as it may lead to a condition known as macrosomia, otherwise known as a “fat” baby, This condition may cause a multitude of problems, including: shoulder damage during birth due to their size, low blood glucose (hypoglycemia) at birth, and respiratory problems. The adverse effect may persist throughout the baby's life, leading to increased risk of childhood obesity and Type-2 diabetes as an adult.

Another major complication that affects pregnancies is preeclampsia (PE). The Mayo Clinic defines PE as “high blood pressure and excess protein in the urine after 20 weeks of pregnancy in a woman who previously had normal blood pressure” (Mayo Clinic Staff “Diseases and Conditions: Preeclampsia” Mayo Clinic). This condition is pregnancy specific, and also contributes considerably to morbidity and mortality of bath the mother and the fetus, as it is the leading global cause of maternal death. Moreover, this condition can have adverse effects for women later on in life, as it increases risk of hypertension, coronary artery disease, stroke, and Type-2 diabetes. As with PTB, gestational diabetes, and many other pregnancy complications, the causes of preeclampsia are not fully understood. The incidence of PE is about 5% for primigravida, or a woman's first pregnancy, and has a global incidence of about 40 million women each year.

To date, there have been very few studies (academic or commercial) that have looked at pregnancy complications, namely preterm birth, at a multifactorial level. The literature is filled with examples looking at 1 or 2 factors for preterm birth, and few that include heterogeneous factors. The first major attempt at making sense of PTB on a multifactorial level was attempted by the Institute of Medicine (IOM), which spanned the course of several years. The objective of the study was to look at PTB on a multifactorial level and study the influences of factors such as: “individual level behavioral and psychosocial factors, neighborhood characteristics, environmental exposures, medical conditions, infertility treatments, biological factors, and genetics,” and a combination of the factors (Institute of Medicine of the National Academies. Preterm Birth:Causes, Consequences, and Prevention. Wash. D.C.: The National Academies Press, 2007). The authors of this study, in an attempt to make sense of the complexity of this condition, were able to pinpoint key topics of interest, intended to help guide the study. In doing so they were able to identify three key areas: “(1) the establishment of multidisciplinary research centers; (2) improved research in three areas including better definition of the problem of preterm birth with improved data, clinical and health services research investigations, and etiologic and epidemiologic investigations; and (3) the study and informing of public policy” (Institute of Medicine of the National Academies. Preterm Birth: Causes, Consequences, and Prevention. Wash. D.C.: The National Academies Press, 2007; incorporated herein by reference in its entirety).

In spite of all the time and efforts spent trying to understand PTB, gestational diabetes, PE, and other pregnancy complications, the underlying causes still remain unclear. There is an urgent need for identification of the causes of these pregnancy complications, and development of interventions. This remains a gap in the healthcare system that has yet to be filled.

### SUMMARY

The present disclosure generally relates to devices and methods for reducing the risk of preterm-labor and preterm-birth. The present disclosure also generally relates to the field of delivering therapeutically effective amounts of medicaments, facilitated by medical devices described herein, for use in treating diseases, disorders, or conditions in a subject mammal.

Accordingly, provided herein is a medical device for reducing the risk of preterm-labor and preterm-birth. In some embodiments, the medical device includes a biocompatible cervical implant and a biocompatible conduit. In some embodiments, the biocompatible cervical implant and the biocompatible conduit are operably linked to allow passage of matter through the medical device. In some embodiments, the biocompatible cervical implant or the biocompatible conduit include at least one medicament. In some embodiments, the biocompatible cervical implant is a cervical barrier, a cervical plug, a cervical stent, or a cervical ring.

In some embodiments, the biocompatible cervical implant or the biocompatible conduit is manufacture from or includes a material including, for example, polylactic acid (PLA), poly(lactic-co-glycolic acid) (PLGA), polyglycolic acid (PGA), polyethylene glycol (PEG), polycaprolactone (PCL), polydioxanone (PDO), poly(lactide-co-glycolide), polyglyconate, polyhydroxybutyrate (PHB), polyhydroxyvalerate (PHV), poly-pseudo amino acids, polyesteramides, silk cocoons or silk derivatives, polyanhydrides, polyorthoesters, polyphosphazones, thermoplastic elastomers, polyether block amide (PEBA), fluoropolymers, silicone, latex or hypoallergenic latex, all materials listed on FDA-approved Generally Recognizes As Safe (GRAS) list, ethylenedia.minetetraacetic acid (EDTA), animal epithelia or non-immunogenic collagens, nano-materials, or a combination thereof.

In some embodiments, the biocompatible cervical implant or the biocompatible conduit includes one or more biodegradable materials. In some embodiments, the biocompatible cervical implant or the biocompatible conduit includes one or more non-biodegradable materials.

In some embodiments, the biocompatible conduit includes at least one one-way valve facing away from the cervix when placed within a subject. In some embodiments, the at least one one-way valve is a bicuspid valve, a multicuspid valve, a globe valve, a ball valve, a needle valve, a pinch valve, a relief valve, or a check valve.

In some embodiments, the matter is a bodily fluid, secretion, bacteria, toxin, or foreign matter.

In some embodiments, the medicament includes a therapeutically effective amount of a therapeutic agent selected from an antimicrobial, a chemotherapy, a hormonal therapy, an antiviral, a selective bacterio-therapy compositions, or a combination thereof. In some embodiments, the antimicrobial includes a therapeutically effective amount of amoxicillin, ampicillin, clindamycin, erythromycin, metronidazole, penicillin, gentamicin, ampicillin, sulbactam, cefoxitin, cefotetan, cefazolin, ciprofloxacin, penicillin G, penicillin V, rifampin, vancomycin, azithromycin, ampicillin-sulbactam, ampicillian-clavulanic acid, cephalosporins, levofloxacin, quinolones, chloramphenicol, nitrofurantoin, cefuroxime, cefaclor, cefadroxil, cefamandole, cefpodoxirne, cefprozil, ceftazidime, ceftibuten, ceftizoxime, ceftriaxone, cephalexin, cephapirin, cephradine, cefdinir, cefditoren, cefepime, cefixime, sulfamethoxazole I trimethoprim, imidazole, triazole, thiazole, antinematodes, anticestodes, antitrematodes, antiamoebics, antiprotozoals, or a combination thereof. In some embodiments, the chemotherapy includes a therapeutically effective amount of cisplatin, carboplatin, paclitaxel, topotecan, gemcitabine, docetaxel, ifosfamide, 5-fluorouracil (5-FU), irinotecan, mitomycin, or a combination thereof. In some embodiments, the hormonal therapy includes a therapeutically effective amount of estrogen and derivatives thereof, progesterone and derivatives thereof, aldosterone and derivatives thereof, steroids and derivatives thereof, or a combination thereof. In some embodiments, the antiviral includes a therapeutically effective amount of vaccines, including influenza vaccinations. In some embodiments, the selective bacterio-therapy composition is a therapeutically effective amount of beneficial aerobic bacteria, such as a lactic-acid bacteria.

In some embodiments, the medicament includes one or more of activated charcoal, copper based compounds, silver based compounds, nickel-based compounds, zinc based compounds, titanium dioxide, or a combination thereof. In some embodiments, the medicament includes histamine and antihistamine therapies. In some embodiments, the medicament includes aptamers. In some embodiments, the medicament includes peptide nucleic acids. In some embodiments, the medicament includes molecular imprinted biopolymers or chemical polymers.

Also provided is a method of using the device to treat, prevent, or reduce the risk of a pregnancy complication, in some embodiments, the method includes selecting a subject in need, providing a medical device as described herein, and operably engaging the medical device with the subject in need. In some embodiments, the subject in need is thereby treated for at least one disease, disorder, or condition.

In some embodiments, the medical device includes at least one biocompatible conduit. In some embodiments, the at least one biocompatible conduit permits substantially one-way flow of matter through the medical device. In some embodiments, the at least one biocompatible conduit includes at least one one-way valve permitting flow through of matter, bodily fluids, or a combination thereof. In some embodiments, the at least one biocompatible conduit prevents infection of the chorion, amnion, amniotic fluid, or a combination thereof, by preventing the inward invasion by any bacterial species of the chorion, amnion, or amniotic fluid. In some embodiments, the at least one biocompatible conduit prevents Intra Amniotic Infection (IAI), initiation of inflammatory cascades, which lead to preterm birth (PTB), other pregnancy complications, or a combination thereof.

In some embodiments, the subject in need is a human primate, a non-human primate, a research animal, a test animal, a veterinary animal, a food-source animal, an agricultural animal, a companion animal, an exotic animal, a human patient, or non-human patient.

In some embodiments, the medical device adheres to the cervical surface of the subject in need. In some embodiments, the medical device encircles the cervix of the subject in need. In some embodiments, the medical device protects the integrity of the cervical mucus plug of the subject in need. In some embodiments, the medical device prevents invasion of microbes through the cervical opening of the subject in need. In some embodiments, the medical device prevents infection of at least one biological tissue of the subject in need. In some embodiments, the at least one biological tissue includes cervix and cervical tissues, vagina and vaginal epithelium, endometrium, uterine myometrial and decidual layers, placental, amniotic and chorionic compartments, or a combination thereof.

In some embodiments, treating a subject in need protects against preterm labor (PTL), preterm birth (PTB), other pregnancy complications, or a combination thereof of the subject in need. In some embodiments, treating a subject in need includes administration of therapeutics for at least one disease, disorder or condition in the subject in need.

In some embodiments, the at least one disease, disorder or condition includes cervical cancer, hormonal imbalances, fungal infections, bacterial vaginosis, chronic inflammatory diseases, or a combination thereof. In some embodiments, the disease, disorder or condition include, but are not limited to preterm labor, preterm birth, intraamniotic infection, cervical cancer, bacterial vaginosis, yeast infection, hormonal imbalances, viral infections, inflammation or chronic inflammation, or a combination thereof.

In some embodiments, treating a subject in need includes, but is not limited to, impregnation of medicament within the medical device, coating medicament onto the medical device, application of medicament by subject in need, or a medical practitioner, in, on, or around the medical device, distribution of medicament via the conduit of the medical device, promoted by dripping of medicament through the conduit due to pressure, or a combination thereof.

In some embodiments, treating a subject in need includes having at least one receptacle designed to house and deliver at least one part of medicament. In some embodiments, treating a subject in need includes pulsatile release of medicament from an electronically controlled reservoir within the medical device. In some embodiments, treating a subject in need includes administration of beneficial selective bacteria-therapies or defined pro biotic mixtures, which promote neonatal digestion of lactal/lacteal proteins during nursing.

In some embodiments, the method of using the medical device to treat a subject in need results in regrowth of the cervical mucus plug of the subject in need.

In some embodiments, operably engaging the medical device includes adhesion to the cervical surface by suction pressure, use of non-suture biocompatible adhesive compositions or chemicals, Velcro-like surfaces within the medical device gripping the outer cervix, vacuum-assisted wound-closure mechanisms, inward facing nano-surfaces that adhere to the cervix, or a combination thereof. In some embodiments, operably engaging the medical device includes encircling of the medical device around the cervix, fastening onto it.

These features, together with other features herein further explained, will become obvious through a reading of the following description of the drawings and detailed description

### BRIEF DESCRIPTION OF THE DRAWINGS

FIGS. 1A and 1B depict one embodiment of a biocompatible cervical barrier of the present disclosure. FIG. 1A depicts a side view, and FIG. 1B depicts a top view.

FIG. 2 depicts the placement of a biocompatible cervical barrier described herein within the cervical canal.

FIG. 3A depicts one embodiment of a biocompatible cervical ring described herein. FIG. 3B depicts the placement of the cervical ring within the cervical canal.

FIGS. 4A and 4B depict one embodiment of a biocompatible intra-cervical plug of the present disclosure; FIG. 4A shows a lateral view of one embodiments of the intra-cervical plug, and FIG. 4B depicts the placement of the plug within the cervical canal.

FIGS. 5A and 5B depict one embodiment of a biocompatible intra-cervical stent described herein. FIG. 5A depicts a lateral view and FIG. 5B depicts an end view.

FIG. 6 depicts the placement of an intra-cervical stent described herein within the cervical canal.

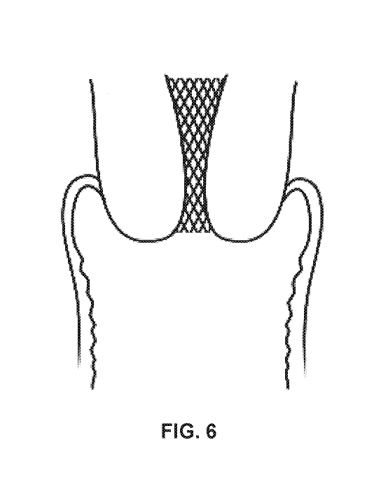

### DETAILED DESCRIPTION

Unless defined otherwise, all technical and scientific terms used herein have the same meaning as commonly understood by one of ordinary skill in the art to which this disclosure belongs. Generally, the nomenclature used herein and the laboratory procedures in cell culture, chemistry, microbiology, molecular biology, cell science and cell culture described below are well known and commonly employed in the art. Conventional methods are used for these procedures, such as those provided in the art and various general references such as Preterm Birth: Causes, Consequences, and Prevention, Richard E. Behrman, Adrienne Stith Butler, Editors, Committee on Understanding Premature Birth and Assuring Healthy Outcomes ISBN: 0-309-65 898-5, 790 pages, 6×9, (2007), and Behrman. R E and Butler, A S, Editors. Preterm Birth: Causes. Consequences, and Prevention Institute of Medicine of the National Academies, 2007. The National Academy Press, Wash., D.C.

Where a term is provided in the singular, the plural of that term is also contemplated. The nomenclature used herein and the laboratory procedures described below are those well-known and commonly employed in the art. As employed throughout the disclosure, the following terms, unless otherwise indicated, shall be understood to have the following meanings:

As it is used in the present disclosure the term biomarkers is defined in the same way as The National Institutes of “Health Biomarkers Definitions Working Group” which defines a biomarker as: “A characteristic that is objectively measured and evaluated as an indicator of normal biological processes, pathogenic processes, or pharmacologic responses to a therapeutic intervention.”

The terms “assessing” and “diagnosing” as used herein refer, in general, to an evaluation by a physician or other health care provider, who can conduct an assessment that provides a preliminary evaluation as to the disease or injury affecting a subject, and diagnostic tests provide (in many cases but not always) the diagnosis in the results, or can require further diagnostic tests. Diagnostic tests are used to plan, implement care, or to confirm a pre-diagnosis or in some cases diagnostic tests can be used for a prognosis. Lab testing after the diagnosis can be used to verify that the diagnosis is correct, or lab tests can be used to verify the accuracy of other tests or used to monitor a treatment or for prognosis. The tests are used for assessing risks and as a diagnostic tool as a physician or health care provider, such as but not limited to an OB/Gyn, can carry out other routine tests and assessments.

As used herein, the term “pregnancy cervical mucus plug” generally refers to the specialized copious thick mucus that fills the endocervical canal during pregnancy and protects against vertical invasion of vaginal bacteria into the fetal/placental compartment.

As used herein, the term “cervical length” generally refers to the vaginal ultrasonic measurement of the length of the endocervical canal from the internal orifice of the uterus (internal os) to the external orifice of the uterus (external os), A short cervix before 28 weeks gestation generally refers to a cervical length of less than 2.5 cm.

As used herein, the term “cervical canal” refers to the internal cervical passageway which extends from the external cervical os in the vagina to the internal cervical os at the beginning of the lower uterine segment. During pregnancy, the internal cervical os abuts the placental amnion and chorion, which contain the amniotic fluid and the fetus.

Other technical terms used herein have their ordinary meaning in the art that they are used, as exemplified by, but not limited to, a variety of technical dictionaries.

The present disclosure recognizes that there exists a need for treating pregnancy complications, including but not limited to preterm labor and preterm birth. The present disclosure also recognizes that there is a need for treating diseases, disorders, or conditions in a subject mammal, as well in the fetus when the subject mammal is pregnant. The present disclosure generally describes medical devices, and methods of use for these devices, which are used to reduce the risk of preterm-labor and preterm-birth. This disclosure also generally relates to the field of delivering therapeutically effective amounts of medicaments, facilitated by these medical devices, for use in treating diseases, disorders, or conditions in a subject mammal and its fetus.

Accordingly, provided herein are devices for reducing the risk of preterm-labor. The several embodiments and technical features of the devices are generally described as the following, each of which will be described in greater detail: biocompatible cervical barriers; biocompatible intra-cervical plugs; biocompatible intra-cervical stents; and biocompatible vaginal rings. In addition, provided herein are methods for using the medical devices described herein, wherein the methods include: methods of using a biocompatible cervical barrier; methods of using a biocompatible intra-cervical plug; methods of using a biocompatible intra-cervical stent; and methods of using a biocompatible vaginal ring. A person of skill in the art will understand that the devices and methods as described herein are non-limiting, but are provided as exemplary features and aspects of devices for treating, ameliorating, or preventing pregnancy complications.

In some embodiment is provided a medical device, including: (a) at least one biocompatible cervical barrier, including a first surface and a second surface; and (b) at least one biocompatible conduit; wherein the at least one cervical barrier and the at least one conduit are operably linked to allow passage of matter through the medical device; and wherein the at least one biocompatible cervical barrier, the at least one biocompatible conduit, or a combination thereof, include at least one medicament.

In some embodiments is provided a method for treating a subject mammal in need for at least one disease, disorder or condition, including: (a) providing a medical device of the present disclosure including a cervical barrier; and (b) operably engaging the medical device with the subject mammal in need; wherein the subject mammal in need is treated for at least one disease, disorder or condition; wherein the conduit permits substantially one-way flow through of matter; and optionally wherein the medical device promotes regrowth of the cervical mucus plug of the subject mammal in need.

In some embodiments is provided a medical device, including: (a) at least one biocompatible intra-cervical plug, including a first surface and a second surface; and (b) at least one biocompatible conduit; wherein the at least biocompatible one intra-cervical plug and the at least one conduit are operably linked to allow passage of matter through the medical device; wherein the at least one biocompatible intra-cervical plug, the at least one biocompatible conduit, or a combination thereof, include at least one medicament.

In some embodiments is provided a method for treating a subject mammal in need for at least one disease, disorder or condition, including: (a) providing a medical device including an intra-cervical plug; and (b) operably engaging the medical device with the subject mammal in need; wherein the subject mammal in need is treated for at least one disease, disorder or condition; and optionally wherein the medical device promotes regrowth of the cervical mucus plug of the subject mammal in need.

In some embodiments is provided a medical device, including: at least one biocompatible intra-cervical stent, including a first surface and a second surface, which can be optionally designed to trap mucus or retard its outflow to the vagina; wherein the at least one biocompatible intra-cervical stent includes at least one medicament.

In some embodiments is provided a method for treating a subject mammal in need for at least one disease, disorder or condition, including: (a) providing a medical device including an intra-cervical stent; and (b) operably engaging the medical device with the subject mammal in need; wherein the subject mammal in need is treated for at least one disease, disorder or condition; and optionally wherein the medical device promotes regrowth of the cervical mucus plug of the subject mammal in need.

In some embodiments is provided a medical device, including: at least one biocompatible vaginal ring, wherein the at least one biocompatible vaginal ring includes at least one medicament.

In some embodiments is provided a method for treating a subject mammal in need for at least one disease, disorder or condition, including: (a) providing a medical device including a vaginal ring; and (b) operably engaging the medical device with the subject mammal in need; wherein the subject mammal in need is treated for at least one disease, disorder or condition.

These aspects of the disclosure, as well as others described herein, can be achieved by using the methods, articles of manufacture, and compositions of matter described herein. To gain a full appreciation of the scope of the present disclosure, it will be further recognized that various embodiments of the present disclosure can be combined to make desirable embodiments of the disclosure.

The present disclosure generally pertains to the field of treating pregnancy complications, such as but not limited to preterm labor and preterm birth. More specifically the present disclosure pertains to protecting the cervical mucus plug of a subject pregnant mammal. More specifically, the present disclosure aims to develop medical devices for protecting the cervical mucus plug of a mammal, promoting regrowth of the mucus plug. More specifically, the present disclosure provides a method of administering a medicament for treating pregnancy complications, and other diseases, disorders or conditions. More specifically still the pregnancy complications include, but aren't limited to, women who are at an increased risk of damage to or degradation of the cervical mucus plug, vaginal microbiome dysbiosis, bacterial vaginosis (BV), vaginitis, recurrent spontaneous abortions (RSA), preterm birth, gestational diabetes, pre-eclampsia including hemolysis elevated liver enzymes low platelets (HELLP Syndrome), preterm premature rupture of the membranes (PPROM), premature rupture of the membranes (PROM), antepartum hemorrhage including placental abruption, chorioamnionitis, intrauterine growth restriction, placenta previa, and sequelae of intraamniotic infection such as inflammatory cytokine cascades leading to premature contractions and birth. Each of the embodiments described above are described in greater detail below.

### Biocompatible Cervical Barrier

In some embodiments is provided a medical device, including: a) at least one biocompatible cervical barrier, including a first surface and a second surface; and b) at least one biocompatible conduit; wherein the at least one cervical barrier and the at least one conduit are operably linked to allow passage of matter through the medical device; and wherein the at least one biocompatible cervical barrier, the at least one biocompatible conduit, or a combination thereof, include at least one medicament.

As used herein, the term “biocompatible” refers to a material that may be used in a living organism and that will not be rejected by the organism. Thus, in some embodiments, the at least one biocompatible cervical barrier, the at least one biocompatible conduit, or both, can include and may or may not be biodegradable or biodegradable: polylactic acid (PLA), poly-lactic-co-glycolic acid (PLGA), polyglycolic acid (PGA), polyethalene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons, polyanhydrides, polyorthoesters, polyphosphazones, thermoplastic elastomers, polyether block amide (PEBA), fluoropolymers, silicone, latex, animal epithelia, spider or cocoon silk, nano-materials, poly(ethylene-covinyl acetate), hydrophilic polyether urethane (HPEU) polyether urethane (PEU), osmotic agents (e.g. NaCl or sodium acetate), polyhydroalkanoates (PHAs), such as polyhdroxyhexanoate (PHH), polybutylene succinate (PBS), polycaprolactone (PCL), cellulose esters, GD polyvinyl alcohol, or a combination thereof.

Additional material include but are not limited to all FDA-approved and Generally Recognized As Safe (GRAS) materials, including but not limited to EDTA, or non-immunogenic or immunogenic collagens such as but not limited to collagens such as bovine collagen, either alone or in combination with other materials.

In some embodiments, the at least one biocompatible cervical barrier, the at least one biocompatible conduit, or both, include one or more biodegradable materials.

In some embodiments, the at least one biocompatible cervical barrier includes a substantially a dome shape. The dome shape allows the barrier to fit under the cervix like a cup, allowing the device to substantially or completely cover and optimally protect the cervix and to gather mucus and secretions.

In some embodiments, the at least one biocompatible cervical barrier includes a substantially a flat shape. The flat shape of the device can allow the device to better cover and protect the cervix in anatomical situations where the dome shape could in some instances be less suitable in degree for this purpose.

In some embodiments, the at least one biocompatible cervical barrier includes a substantially a circular vaginal ring shape. The vaginal ring may be manufactured in many different cross-sectional diameters and degrees of rigidity or elasticity of material. The ring may be designed to provide structural support to the cervix, or to provide delivery of medicaments to or through the vaginal or cervical epithelium, or a combination of both functions, The vaginal ring will be open in the middle, thus allowing for spontaneous drainage of cervical and vaginal secretions through the ring to the lower vagina.

In some embodiments, the at least one biocompatible cervical barrier includes at least in part at least one rim. The rim or rims of the barrier preferably and optionally assist the device to seat firmly against the vaginal walls and hold the device in place. Sturdy rims can allow the remainder of the device to be less bulky, for better comfort of the subject.

In some embodiments, the at least one biocompatible cervical barrier is at least in part elastic. The elastic quality of the cervical barrier can allow the device to accommodate to the muscular walls of the vagina, and to optionally change shape as the vaginal walls change in response to sexual intercourse or physical exercise.

In some embodiments, the at least one biocompatible cervical barrier is at least in part resilient. The resilient quality of the device can allow the device to spring back into normal position after its shape has changed to conform to changes in the vaginal wall,

In some embodiments, the first surface is designed to face away from the cervix when placed within a subject.

In some embodiments, the first surface is substantially convex.

In some embodiments, the first surface has protrusion housing the conduit, or the conduit or valve may be flush with the first surface

In some embodiments, the second surface is designed to face toward the cervix when placed within a subject.

In some embodiments, the second surface is substantially concave.

In some embodiments, the second surface has a perforation leading to the conduit.

In some embodiments, the concave surface can be used as a reservoir to store any medicaments or antibiotics to prevent cervical or chorionic infection of the mammal in need but should preferably not interfere with any conduit, if the conduit is needed, as the case may be. The concave surface may also be impregnated or coated with FDA-approved medicaments suitable for use in any pregnant mammal in need or its fetus.

In some embodiments, the at least one biocompatible conduit includes at least one one-way valve facing away from the cervix when placed within a subject. These conduit(s) can be of varying diameters, lengths and flexibilities, depending upon the need. The conduit can be designed to carry matter away from the cervix into the lower vagina, while preventing the flow of secretions, bacteria, toxins or foreign matter up to the cervix. The nature of the valve can be determined by the nature of the matter to be released and the nature of the matter to be excluded particular to the kind of mammal and whether the mammal is pregnant or not.

In some embodiments, the at least one one-way valve includes a bicuspid valve, a multicuspid valve, a ball valve, a needle valve, a pinch valve, a plug valve, a relief valve, or a check valve.

In some embodiments, the at least one biocompatible conduit permits intracervical pressure-activated passage of matter through the medical device outwards towards the vaginal compartment.

In some embodiments, the at least one biocompatible cervical barrier and the at least one biocompatible conduit are operably linked at the apex of the medical device. In some embodiments, the at least one biocompatible cervical barrier, and the at least one biocompatible conduit are operably linked within the protrusion. In some embodiments, the at least one biocompatible cervical barrier, and the at least one biocompatible conduit are operably linked within the perforation on the second surface.

In some embodiments, the medicament includes a therapeutically effective amount of therapeutic agents including: antibiotics, antifungals, antivirals, anti-parasitics, chemotherapies, hormonal therapies, defined selective bacteria-therapy compositions, vaccines, activated charcoal, metal-based compounds, histamines and anti-histamines, immuno-interactive and immunomodulatory agents, hydrogels comparable to the pregnant cervical mucus plug, agents to stimulate restoration of the normal cervical mucus plug in pregnancy, biogels with innate antibacterial properties, tocolytics, anti-oxytotics, pharmaceutically acceptable and safe-for-human-use diluents or carriers, or a combination thereof.

The medicaments can be applied to a reservoir, either open or sealed, in the first or second surface of the device, or both, or impregnated within its biocompatible or biodegradable material, and may include one or more medicaments of the same class or different classes.

The technology for enhanced delivery of medicaments to the vagina and cervical mucosa has been documented in the literature (Cu, Yen, and W. Mark Saltzman. “Controlled surface modification with poly (ethylene) glycol enhances diffusion of PLGA nanoparticles in human cervical mucus.” Molecular Pharmaceutics 6.1 (2008): 173-181; Ensign, Laura M., et al. “Mucus penetrating nanoparticles: biophysical tool and method of drug and gene delivery.” Advanced Materials 24.28 (2012): 3887-3894; Navath, Raghavendra S., et al. “Injectable PAMAM dendrimer—PEG hydrogels for the treatment of genital infections: formulation and in vitro and in vivo evaluation.” Molecular Pharmaceutics 8.4 (2011): 1209-1223). These advances in delivery modalities appear to be ideally suited to use with subject device.

The cervical and vaginal mucosa also present an attractive site for delivery of vaccines to induce active immunity. Kuo-Haller et al, reported on favorable IgG and IgA induction in mice via the vaginal rout compared to other routes (Kuo-Haller, Patricia, et al. “Vaccine delivery by polymeric vehicles in the mouse reproductive tract induces sustained local and systemic immunity.” Molecular Pharmaceutics 7.5 (2010): 1585-1595). In some embodiments, the use of vaccines introduced by subject devices induce antibodies against aberrant components of the immune cascade.

In some embodiments, medicaments include delivery by subject device of selective I-kB (IKK) inhibitors to interfere with local cytokine production in order to prevent or treat preterm labor (De Silva, D., M, D. Mitchell, and J. A. Keelan. “Inhibition of choriodecidual cytokine production and inflammatory gene expression by selective I-κB kinase (IKK) inhibitors.” British Journal of Pharmacology 160.7 (2010): 1808-1822).

In some embodiments, medicaments in this disclosure includes delivery by subject device of appropriately designed synthetic small interfering RNA to influence targeted gene DNA expression of cytokines or other elements of the immune cascade involved in preterm labor. Recent papers have described many ways that such siRNMs can be protected for delivery to cells and delivered vaginally (Singha, Kaushik, Ran Namgung, and Won Jong Kim, “Polymers in small-interfering RNA delivery.” Nucleic Acid Therapeutics 21.3 (2011): 133-147; Woodrow, Kim A., et al. “Intravaginal gene silencing using biodegradable polymer nanoparticles densely loaded with small-interfering RNA.” Nature Materials 8.6 (2009): 526-533).

In some embodiments, the antimicrobials include a therapeutically effective amount of pregnancy-safe therapeutic antibiotics including: amoxicillin, ampicillin, clindamycin, erythromycin, metronidazole, penicillin, gentamicin, ampicillin-sulbactam, cefoxitin, cefotetan, cefazolin, ciprofloxacin, rifampin, vancomycin, azithromycin, ampicillin-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime. ceftriaxone, cephalexin, cephapirin, cephradine, cefdinir, cefditoren, cefepime, cefixime, sulfamethoxazole/trimethoprim, or a combination thereof.

Systemically applied antibiotics are often ineffective at treating vaginal bacterial infections in the non-pregnant state or ascending infections in pregnancy which reach the uterine decidua or placental amnion and chorion, where they initiate the immune cascade which triggers preterm labor. Clinical trials using both oral and intravaginal antibiotics to prevent preterm birth have been disappointing (McDonald, Helen Margaret, et al., “Antibiotics for treating bacterial vaginosis in pregnancy.” The Cochrane Database of Systematic Reviews 1.1 (2007)). In some embodiments, the devices described herein more effectively deliver antibiotics directly to the vaginal and cervical mucosa by direct proximity and slow release of the antibiotic or antibiotics. In some embodiments, the antifungals include a therapeutically effective amount of antifungal agents including: imidazole, triazole, thiazole, or similar agents or a combination thereof. Vaginal candida infections are commonly treated with antifungal suppositories and creams. The effectiveness of these modes of delivery is compromised by the tendency of the medication to drain out of the vagina. In some embodiments, the subject device of the disclosure creates a slow, sustained, release of antifungals directly into the area of infection which will improve the cure rate and reduce the recurrence rate of these fungal infections.

In some embodiments, the antivirals include a therapeutically effective amount of agents such as but not limited to acyclovir, tenofovir, interferon, imiquimod, or similar agents. Slow, sustained local cervical-vaginal release of therapeutic levels of these agents from the subject device can be efficacious to treat viruses such as but not limited to herpes simplex virus, HIV, and human papilloma virus. The delivery of imiquimod to treat cervical condyloma and HPV-related cervical dysplasia is preferable.

In some embodiments, the antimicrobials include a therapeutically effective amount of therapeutic anti-parasitic agents including antinematodes, anticestodes, antitrematodes, antiamoebics, antiprotozoals, or similar agents, or a combination thereof. The vagina can provide a reservoir fur water-born parasites that is resistant to oral anti-parasitic therapies. The subject device can provide localized treatment.

In some embodiments, the chemotherapies include a therapeutically effective amount of chemotherapies including but not limited to: cisplatin, carboplatin, paclitaxel, topotecan, gemcitabine, docetaxel, ifosfamide, 5-fluorouracil (5-FU), irinotecan, mitomycin, or a combination thereof. Cervical cancer is preceded by progressive degrees of cervical dysplasia. Traditional treatments for dysplasia involve surgical removal of part of the cervix, which may lead to incompetent cervix, late abortion and preterm birth. The sustained local chemotherapy delivery by the subject device and methods to treat cervical dysplasia and thereby prevent cancer and preserve fertility is an attractive application of the present disclosure.

In some embodiments, the hormonal therapies include a therapeutically effective amount of hormonal therapies including but not limited to: estrogen and derivatives thereof, progesterone and derivatives thereof, aldosterone and derivatives thereof, steroids and derivatives thereof, or a combination thereof. Vaginally applied estrogen cream and eluting ring (e.g. Estring™) are currently used to treat cervical-vaginal atrophy. It is reported that “sex hormones and their duration of exposure affect the composition and stability of the microbiome as well as systemic and mucosal immune response” (Brotman, Rebecca M., et al. “Microbiome, sex hormones, and immune responses in the reproductive tract: challenges for vaccine development against sexually transmitted infections.” Vaccine 32.14 (2014): 1543-1552). The present disclosure envisions that co-therapy with hormones by the subject device can improve the efficacy of the other medicaments.

Progesterone delivery by the subject device and methods of the present disclosure can improve efficacy of vaginal progesterone therapy. Progesterone therapy has a documented role in reducing the risk of preterm birth in certain categories of patients. Treatment is by intramuscular 17alpha-hydroxyprogesterone caproate or vaginal natural progesterone gel or capsules. The efficacy of current treatment is suboptimal. (Committee on Practice Bulletins—Obstetrics. Practice Bulletin Number 130: Predication and Prevention of Preterm Birth. Washington: The American College of Obstetricians and Gynecologists. Obstetrics and Gynecology, 2012, Print). In some embodiments, improved sustained local delivery of this hormone to the cervix and uterus by the subject device, possibly utilizing customized hydrogels or nanotechnology, improves treatment.

In some embodiments, the defined selective bacterio-therapy compositions include a therapeutically effective amount of beneficial bacteria, including lactic acid-producing or other bacteria. The healthy vaginal bacterial microbiome is known to be dominated by lactobacillus species and lesser numbers of a diverse group of anaerobic and facultative bacteria (Linhares, Iara M., et al. “The vaginal microbiome: New findings bring new opportunities.” Drug Development Research 74.6 (2013): 360-364). Imbalances in this microbiome can lead to pathological conditions, such as preterm delivery (Ganu, Radhika S., Jun Ma, and Kjersti M. Aagaard. “The role of microbial communities in parturition: is there evidence of association with preterm birth and perinatal morbidity and mortality?” American Journal of Perinatology 30.08 (2013): 613-624; Hyman, Richard W., et al. “Diversity of the vaginal microbiome correlates with preterm birth.” Reproductive Sciences 21.1 (2013): 32-40) or post-menopausal vulvovaginal atrophy (Brotman, Rebecca M., et al. “Association between the vaginal microbiota, menopause status and signs of vulvovaginal atrophy.” Menopause 21.5 (2014): 450-458). In some embodiments, a device described herein provides a reservoir of beneficial bacteria to promote a healthy vaginal microbiome in order to prevent or treat preterm birth, vulvovaginal atrophy, or other diseases or conditions.

In some embodiments, vaccination therapy includes vaccines, such as but not limited to, those against influenza, TdaP, Varicella, HPV Zoster, MMR, Pneumococcal, Meningococcal, Hepatitis A, Hepatitis B. Favorable active immunization can be achieved via the vaginal route compared to other routes. Therefore, an additional application for existing vaccines would be via the subject cervical barrier device, possibly including specialized gels or nanotechnology preparations to increase absorption of the vaccines.

In some embodiments, the medicament includes activated charcoal. Activated charcoal has a strong negative electrical charge along with very fine pores which trap bacteria and toxins, preventing the ascent of bacteria up the endocervical canal and absorbing bacterial endotoxins or other harmful metabolites.

In some embodiments, the medicament includes metal based compounds, such as copper-based compounds, silver-based compounds, nickel-based compounds, zinc-based compounds, or a combination thereof. These metal-based compounds have been shown to be safe in human use in such devices as the copper intrauterine contraceptive device, Essure™ contraceptive implants, and specialized dressings for severe skin burns. These metals have antibiotic activity and may prove efficacious in preventing pathological bacteria from ascending the cervical canal, perhaps in combination with other medicaments.

In some embodiments, the medicament includes titanium dioxide as an anti-microbial and optionally a pigment.

In some embodiments, the medicament includes histamine and antihistamine therapies. Because activation of the immune cascade is tied to preterm one embodiment of the present disclosure includes the case in which local cervical-vaginal histamine/antihistamine therapy delivered by the subject cervical barrier device may help prevent preterm labor by interference with or regulation of one or more elements of the immune system.

In some embodiments, the medicament includes immuno-interactive and immunomodulatory agents, especially functional angonists or antagonists to specific cytokines, chemokines, immunocompetent cells, or other elements of the immune system, including antibodies, binding fragments of antibodies, aptamers, antigens, epitopes, peptide nucleic acids, molecular imprinted biopolymers or chemical polymers, oligonucleotides, antisense oligonucleotides, oligodinucleotides, proteins, peptides, peptidomimetics, nucleic acids, carbohydrates, lipids, small molecule compounds, glucocorticoids, cytostatics, anti-metabolites, anti-CD2 antibody or related binding fragment, anti-CD20 antibody, triple helix molecules, antisense-DNA, anti-sense-RNA, ribozyme, iRNA, miRNA, siRNA, shRNA, cyclosporin, tacrolimus, sirolimus, FTY720, small molecule nuclear factor-kappa B inhibitors, ante-sense compounds, peptide nucleic acids (PNAs) or locked nucleic acids (LNAs), or similar chemical or biological agents. Since it is generally accepted that preterm labor is stimulated by pathological perturbations of the immune inflammatory system, the present disclosure includes the case that the subject device can deliver immune-interactive or immunomodulatory agents directly to the cervix, uterus, placental and fetal compartments for modulation of the abnormal process.

In some embodiments, the medicament can include a hydrogel which mimics the naturally occurring pregnancy cervical mucus plug. The mucus plug can deteriorate prematurely in pregnancy and lead to ascending bacterial infection from the vagina. In some embodiments, an artificial mucous can be manufactured and held in place inside the cervix by the subject device (for a study of cervical mucus, see Critchfield, Agatha S., et al, “Cervical mucus properties stratify risk for preterm birth.” PLOS ONE 8.8 (2013): e69528; Becher. Naja, et al. “The cervical mucus plug: structured review of the literature.” Acta Obstetricia et Gynecologica Scandinavica 88.5 (2009): 502-513).

In some embodiments, the medicament can include an agent to stimulate the restoration of the normal cervical mucus in pregnancy. In some embodiments, the subject device could be used to deliver appropriate agonist and antagonist agents to stimulate production of the proper mucus from the endocervical glands.

In some embodiments, the device is capable of delivery of a specialized biogel with innate antibiotic properties, such as the antibacterial activity exhibited by the amine terminated dendrimer released from a degrading specialized gel (Navath, Raghavendra S., et al. “Injectable PAMAM dendrimer—PEG hydrogels for the treatment of genital infections: formulation and in vitro and in vivo evaluation.” Molecular Pharmaceutics 8.4 (2011): 1209-1223).

In some embodiments, the device is capable of delivery of tocolytic agents, such as ritodrine, terbutaline, hexoprenaline, magnesium sulfate, indomethacine and nifedipine or similar agents. Tocolytics suppress muscular contractions of the uterus. Tocolytics are traditionally administered by intravenous, oral or rectal routes. They have suboptimal efficacy and cause significant side effects. In some embodiments, delivery of smaller concentrations of tocolytics directly to the cervix and uterus via the subject device can be more effective with less systemic side effects.

In some embodiments, the device is capable of delivery of an antioxytocic medicament. Pitocin released physiologically by the pituitary gland stimulates uterine contractions. Induction and augmentation of labor via oxytocin intravenous infusion is a standard obstetrical practice In some embodiments, antibodies to oxytocin or to oxytocin receptors are delivered directly to the cervix via the subject device to effectively inhibit labor.

In some embodiments is provided the inclusion of a suitable diluent or carrier material to stabilize and maintain the correct concentration of the medicament.

### Biocompatible Intra-Cervical Plug

In some embodiments is provided a medical device, including: (a) at least one biocompatible intra-cervical plug, including a first surface and a second surface; and (b) at least one biocompatible conduit; wherein the at least biocompatible one intra-cervical plug and the at least one conduit are operably linked to allow passage of matter through the medical device; and wherein the at least one biocompatible intra-cervical plug, the at least one biocompatible conduit, or a combination thereof, include at least one medicament.

In some embodiments, the at least one biocompatible intra-cervical plug, the at least one biocompatible conduit, or both, can include and may or may not be biodegradable or bioerodable: polylactic acid (PLA), poly-lactic-co-glycolic acid (PLGA), polyglycolic acid (PGA), polyethalene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons, polyanhydrides, polyorthoesters, polyphosphazones, thermoplastic elastomers, polyether block amide (PEBA), fluoropolymers, silicone, latex, animal epithelia, spider or cocoon silk, nano-materials, poly(ethylene-covinyl acetate), hydrophilic polyether urethane (HPEU), polyether urethane (PR)), osmotic agents (e.g. NaCl or sodium acetate), polyhydroalkanoates (PHAs), such as polyhdroxyhexanoate (PHH), polybutylene succinate (PBS), polycaprolactone (PCL), cellulose esters, GD polyvinyl alcohol, or a combination thereof.

Additional material include but are not limited to all FDA-approved and Generally Recognized As Safe (GRAS) materials, including but not limited to EDTA, or non-immunogenic or immunogenic collagens such as but not limited to collagens such as bovine collagen, either alone or in combination with other materials.

In some embodiments, the at least one biocompatible intra-cervical plug, the at least one biocompatible conduit, or both, include one or more biodegradable materials

In some embodiments, the at least one biocompatible intra-cervical plug includes a substantially conical shape wherein the apex is preferably inserted into the cervix and the broader base of the plug remains at the entry of the cervix.

In some embodiments, the at least one biocompatible intra-cervical plug includes a substantially cylindrical shape. The shape of the intra-cervical plug to fit an individual mammal can vary depending according to the species and the individual anatomy.

In some embodiments, the at least one biocompatible intra-cervical plug is substantially a hollow shape with, including but not limited to, internal filaments, pilli, nano-materials, or hair-like materials to trap mucus internally generated in the base and more inward regions of the cervix.

In some embodiments, the at least one biocompatible intra-cervical plug can be substantially hollow or contains a conduit for drainage of excess mucus generated in the cervix once the mucus plug regenerates

In some embodiments, the at least one biocompatible intra-cervical plug includes a first open orifice.

In some embodiments, the first open orifice faces the vagina when placed within a subject.

In some embodiments, the at least one biocompatible intracervical plug includes a second open orifice.

In some embodiments, the second open orifice faces the uterus when placed within a subject.

In some embodiments, the at least one biocompatible intra-cervical plug is at least in part elastic. This property enables the intra-cervical plug to conform to an irregular shape of the endocervical canal. The intra-cervical plug can be hygroscopic, or swells upon contact with intra-vaginal and intra-cervical fluids or mucus. This property enables the device to gently anchor in the endocervical canal.

In some embodiments, the at least one biocompatible intracervical plug is at least in part resilient and can be atraumatically removed by a qualified medical or veterinarian professional as deemed appropriate once the mucus plug regenerates sufficiently.

The intra-cervical plug or optionally biodegrades at a substantially equal rate as that of the regeneration of the mucus plug preferably according to the diagnosis of a qualified medical professional that prescribes its necessary or preferable duration of treatment. This obviates or reduces the need to remove the device after it has accomplished its purpose.

In some embodiments, the first surface is designed to face the cervical epithelium when placed within a subject.

In some embodiments, the first surface includes: barbs, prongs, spikes, thistles, bristles, quills, spurs, a biofilm, a micro-abrasive nanosurface with a high coefficient of friction or bio-adhesive surface activated by implantation in the cervix, or combination thereof.

In some embodiments, the first surface is of proper frictional character such that the device is preferably automatically expelled from the cervix once internal pressure from the regenerated mucus plug is sufficient to maintain homeostasis in the cervix and prevent immigration or inwards migration of harmful bacterial species to the upper cervix and chorion. Thus, a conduit may or may not be needed in this device, in some embodiments, the first surface is the outer surface.

In some embodiments, the second surface is designed to face away from the cervical epithelium when placed within a subject. In some embodiments, the second surface is the inner surface. In some embodiments, the second surface houses the at least one biocompatible conduit.

In some embodiments, the intra-cervical plug includes a concave surface. In some embodiments, the concave surface can be used as a reservoir to store any medicaments or antibiotics to prevent cervical or chorionic infection of the mammal in need. The concave surface can be coated with a medicament that is dispensed over time via enzymatic action or solubility, or the concave surface may be impregnated with the medicament that is then dispensed over time via osmosis, bio-entropy or biodegradation of the bioerodable material.

In some embodiments, the at least one biocompatible conduit includes at least one one-way valve facing the first open orifice preferably facing the vaginal compartment. In some embodiments, the at least one one-way valve includes a bicuspid valve, a multicuspid valve or multi-cuspid valve of any number of cuspids, a globe valve, a ball valve, a needle valve, a pinch valve, a plug valve, or a check valve. In some embodiments, the at least one one-way valve includes an optionally pressure-activated relief valve.

In some embodiments, the at least one biocompatible conduit permits passage of mucus, fluids and matter through the medical device, possibly due to pressure internally generated in the upper and middle cervix.

In some embodiments, the at least one biocompatible intra-cervical plug and the at least one biocompatible conduit are operably linked at the second surface.

In some embodiments, the at least one biocompatible intra-cervical plug and the at least one biocompatible conduit are operably linked and are preferably positioned at: the first open orifice, the second open orifice, or an intermediary position thereof.

In some embodiments, the medicament includes a therapeutically effective amount of therapeutic agents including: antibiotics, antifungals, antivirals, anti-parasitics, chemotherapies, hormonal therapies, defined selective bacterio-therapy compositions, vaccines, activated charcoal, metal-based compounds, histamines and anti-histamines, immuno-interactive and immunomodulatory agents, hydrogels comparable to the pregnant cervical mucus plug, agents to stimulate restoration of the normal cervical mucus plug in pregnancy, biogels with innate antibacterial properties, tocolytics, anti-oxytotics, pharmaceutically acceptable and safe-for-human-use diluents or carriers, or a combination thereof.

The medicaments can be applied to a reservoir, either open or sealed, in the first or second surface of the device, or both, or impregnated within its biocompatible or biodegradable material, and may include one or more medicaments of the same class or different classes.

The technology for enhanced delivery of medicaments to the vagina and cervical mucosa has been documented in the literature (Cu, Yen, and W. Mark Saltzman. “Controlled surface modification with poly (ethylene) glycol enhances diffusion of PLGA nanoparticles in human cervical mucus.” Molecular Pharmaceutics 6.1 (2008): 173-181; Ensign, Laura M., et al. “Mucus penetrating nanoparticles: biophysical tool and method of drug and gene delivery.” Advanced Materials 24.28 (2012): 3887-3894; Navath, Raghavendra S., et al, “Injectable PAMAM dendrimer—PEG hydrogels for the treatment of genital infections: formulation and in vitro and in vivo evaluation.” Molecular Pharmaceutics 8.4 (2011): 1209-1223). These advances in delivery modalities appear to be ideally suited to use with subject device.

The cervical and vaginal mucosa also present an attractive site for delivery of vaccines to induce active immunity. Kuo-Haller at al reported on favorable IgG and IgA induction in mice via the vaginal rout compared to other routes (Kuo-Haller, Patricia, et al. “Vaccine delivery by polymeric vehicles in the mouse reproductive tract induces sustained local and systemic immunity.” Molecular Pharmaceutics 7.5 (2010): 1585-1595). In some embodiments, the use of vaccines introduced by subject devices induce antibodies against aberrant components of the immune cascade.

In some embodiments is provided delivery by subject device of selective I-kB (IKK) inhibitors to interfere with local cytokine production in order to prevent or treat preterm labor (De Silva, D., M. D. Mitchell, and J. A. Keelan. “Inhibition of choriodecidual cytokine production and inflammatory gene expression by selective I-κB kinase (IKK) inhibitors.” British Journal of Pharmacology 160.7 (2010): 1808-1822).

In some embodiments is provided delivery by subject device of appropriately designed synthetic small interfering RNA to influence targeted gene DNA expression of cytokines or other elements of the immune cascade involved in preterm labor. Recent papers have described many ways that such siRNMs can be protected for delivery to cells and delivered vaginally (Singha, Kaushik, Ran Namgung, and Won Jong Kim. “Polymers in small-interfering RNA delivery,” Nucleic Acid Therapeutics 21.3 (2011): 133-147; Woodrow, Kim A., et al. “Intravaginal gene silencing using biodegradable polymer nanoparticles densely loaded with small-interfering RNA.” Nature Materials 8.6 (2009): 526-533).

In some embodiments, the antimicrobials include a therapeutically effective amount of pregnancy-safe therapeutic antibiotics including: amoxicillin, ampicillin, clindamycin, erythromycin, metronidazole, penicillin, gentamicin, ampicillin-sulbactam, cefoxitin, cefotetan, cefazolin, ciprofloxacin, rifampin, vancomycin, azithromycin, ampicillin-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazole/trimethoprim, or a combination thereof.

Systemically applied antibiotics are often ineffective at treating vaginal bacterial infections in the non-pregnant state or ascending infections in pregnancy which reach the uterine decidua or placental amnion and chorion, where they initiate the immune cascade which triggers preterm labor. Clinical trials using both oral and intravaginal antibiotics to prevent preterm birth have been disappointing (McDonald, Helen Margaret, et al.. “Antibiotics for treating bacterial vaginosis in pregnancy.” The Cochrane Database of Systematic Reviews 1.1 (2007)). In some embodiments, the subject device to more effectively deliver antibiotics directly to the vaginal and cervical mucosa by direct proximity and slow release of the antibiotic or antibiotics.

In some embodiments, the antifungals include a therapeutically effective amount of antifungal agents including: imidazole, triazole, thiazole, or similar agents or a combination thereof. Vaginal candida infections are commonly treated with antifungal suppositories and creams. The effectiveness of these modes of delivery is compromised by the tendency of the medication to drain out of the vagina. In some embodiments, the subject device of the disclosure can create a slow, sustained release of antifungals directly into the area of infection which will improve the cure rate and reduce the recurrence rate of these fungal infections.

In some embodiments, the antivirals include a therapeutically effective amount of agents such as but not limited to acyclovir, tenofovir, interferon, imiquimod, or similar agents.

Slow, sustained local cervical-vaginal release of therapeutic levels of these agents from the subject device can be efficacious to treat viruses such as but not limited to herpes simplex virus, HIV, and human papilloma virus. The delivery of imiquimod to treat cervical condyloma and HPV-related cervical dysplasia is preferable.

In some embodiments, the antimicrobials include a therapeutically effective amount of therapeutic anti-parasitic agents including antinematodes, anticestodes, antitrematodes, antiamoebics, antiprotozoals, or similar agents, or a combination thereof. The vagina can provide a reservoir for water-born parasites that is resistant to oral anti-parasitic therapies. The subject device can provide localized treatment.

In some embodiments, the chemotherapies include a therapeutically effective amount of chemotherapies including but not limited to: cisplatin, carboplatin, paclitaxel, topotecan, gemcitabine, docetaxel, ifosfamide, 5-fluorouracil (5-FU), irinotecan, mitomycin, or a combination thereof. Cervical cancer is preceded by progressive degrees of cervical dysplasia. Traditional treatments for dysplasia involve surgical removal of part of the cervix, which may lead to incompetent cervix, late abortion and preterm birth. The sustained local chemotherapy delivery by the subject device and methods to treat cervical dysplasia and thereby prevent cancer and preserve fertility is an attractive application of the present disclosure.

In some embodiments, the hormonal therapies include a therapeutically effective amount of hormonal therapies including but not limited to: estrogen and derivatives thereof, progesterone and derivatives thereof, aldosterone and derivatives thereof, steroids and derivatives thereof, or a combination thereof. Vaginally applied estrogen cream and eluting ring (e.g. Estring™) are currently used to treat cervical-vaginal atrophy. It is reported that “sex hormones and their duration of exposure affect the composition and stability of the microbiome as well as systemic and mucosal immune response” (Brotman, Rebecca M., et al. “Microbiome, sex hormones, and immune responses in the reproductive tract: challenges for vaccine development against sexually transmitted infections.” Vaccine 3214 (2014): 1543-1552). In some embodiments, co-therapy with hormones by the subject device improves the efficacy of the other medicaments.

Progesterone delivery by the subject device and methods of the present disclosure can improve efficacy of vaginal progesterone therapy. Progesterone therapy has a documented role in reducing the risk of preterm birth in certain categories of patients. Treatment is by intramuscular 17alpha-hydroxyprogesterone caproate or vaginal natural progesterone gel or capsules. The efficacy of current treatment is suboptimal. (Committee on Practice Bulletins—Obstetrics. Practice Bulletin Number 130: Predication and Prevention of Preterm Birth. Washington: The American College of Obstetricians and Gynecologists. Obstetrics and Gynecology, 2012. Print). In some embodiments, improved sustained local delivery of this hormone to the cervix and uterus by the subject device, possibly utilizing customized hydrogels or nanotechnology, improves the success of this treatment.

In some embodiments, the defined selective bacteria-therapy compositions include a therapeutically effective amount of beneficial bacteria, including lactic acid-producing or other bacteria. The healthy vaginal bacterial microbiome is known to be dominated by lactobacillus species and lesser numbers of a diverse group of anaerobic and facultative bacteria (Linhares, Iara M., et al. “The vaginal microbiome: New findings bring new opportunities.” Drug Development Research 74.6 (2013): 360-364). Imbalances in this microbiome can lead to pathological conditions, such as preterm delivery (Ganu, Radhika S., Jun Ma, and Kjersti M. Aagaard. “The role of microbial communities in parturition: is there evidence of association with preterm birth and perinatal morbidity and mortality?” American Journal of Perinatology 30.08 (2013): 613-624; Hyman, Richard W., et al. “Diversity of the vaginal microbiome correlates with preterm birth.” Reproductive Sciences 21.1 (2013): 32-40) or post-menopausal vulvovaginal atrophy (Brotman, Rebecca M., et al. “Association between the vaginal microbiota, menopause status and signs of vulvovaginal atrophy.” Menopause 21.5 (2014): 450-458). One example of the disclosure would be to use the subject device to provide a reservoir of beneficial bacteria to promote a healthy vaginal microbiome in order to prevent or treat preterm birth, vulvovaginal atrophy, or other diseases or conditions.

In some embodiments, vaccination therapy includes vaccines, such as but not limited to, those against influenza, TdaP, Varicella., HPV, Zoster, MMR, Pneumococcal, Meningococcal, Hepatitis A, Hepatitis B.

Recent research has demonstrated that favorable active immunization can be achieved via the vaginal route compared to other routes. Therefore, an additional application for existing vaccines would be via the subject cervical barrier device, possibly including specialized gels or nanotechnology preparations to increase absorption of the vaccines.

In some embodiments, the medicament includes activated charcoal. Activated charcoal has a strong negative electrical charge along with very fine pores which trap bacteria and toxins, preventing the ascent of bacteria up the endocervical canal and absorbing bacterial endotoxins or other harmful metabolites.

In some embodiments, the medicament includes metal based compounds, such as copper-based compounds, silver-based compounds, nickel-based compounds, zinc-based compounds, or a combination thereof. These metal-based compounds have been shown to be safe in human use in such devices as the copper intrauterine contraceptive device, Essure™ contraceptive implants, and specialized dressings for severe skin burns. These metals have antibiotic activity and may prove efficacious in preventing pathological bacteria from ascending the cervical canal, optionally in combination with other medicaments.

In some embodiments, the medicament includes titanium dioxide, as an anti-microbial and optionally a pigment.

In some embodiments, the medicament includes histamine and antihistamine therapies. Because activation of the immune cascade is tied to preterm labor, one embodiment of the present disclosure includes the case in which local cervical-vaginal histamine/antihistamine therapy delivered by the subject cervical barrier device may help prevent preterm labor by interference with or regulation of one or more elements of the immune system.

In some embodiments, the medicament includes immuno-interactive and immunomodulatory agents, especially functional angonists or antagonists to specific cytokines, chemokines, immunocompetent cells, or other elements of the immune system, including antibodies, binding fragments of antibodies, aptamers, antigens, epitopes, peptide nucleic acids, molecular imprinted biopolymers or chemical polymers, oligonucleotides, antisense oligonucleotides, oligodinucleotides, proteins, peptides, peptidomimeties, nucleic acids, carbohydrates, lipids, small molecule compounds, glucocorticoids, cytostatics, anti-metabolites, anti-CD2 antibody or related binding fragment, anti-CD20 antibody, triple helix molecules, anti-sense-DNA, anti-sense-RNA, ribozyme, iRNA, miRNA, siRNA, shRNA, cyclosporin, tacrolimus, sirolimus, FTY720, small molecule nuclear factor-kappa B inhibitors, ante-sense compounds, peptide nucleic acids (PNAs) or locked nucleic acids (LNAs), or similar chemical or biological agents. Because it is generally accepted that preterm labor is stimulated by pathological perturbations of the immune inflammatory system, the present disclosure includes the case that the subject device can deliver immune-interactive or immunomodulatory agents directly to the cervix, uterus, placental and fetal compartments for modulation of the abnormal process.

In some embodiments, the medicament can include a hydrogel which mimics the naturally occurring pregnancy cervical mucus plug. The mucus plug can deteriorate prematurely in pregnancy and lead to ascending bacterial infection from the vagina, in some embodiments, artificial mucous can be manufactured and held in place inside the cervix by the subject device (for a study of cervical mucus, see Critchfield, Agatha S., et al, “Cervical mucus properties stratify risk for preterm birth.” PLOS ONE 8.8 (2013): e69528; Becher. Naja, et al. “The cervical mucus plug: structured review of the literature.” Acta Obstetricia et Gynecologica Scanclinavica 88.5 (2009): 502-513).

In some embodiments, the medicament can include an agent to stimulate the restoration of the normal cervical mucus in pregnancy. In some embodiments, the subject device delivers appropriate agonist and antagonist agents to stimulate production of the proper mucus from the endocervical glands.

In some embodiments, the device is capable of delivery of a specialized biogel with innate antibiotic properties, such as the antibacterial activity exhibited by the amine terminated dendrimer released from a degrading specialized gel (Navath, Raghavendra S., et al. “Injectable PAMAM dendrimer—PEG hydrogels for the treatment of genital infections: formulation and in vitro and in vivo evaluation.” Molecular Pharmaceutics 8.4 (2011): 1209-1223).

In some embodiments, the device is capable of delivery of tocolytic agents, such as ritodrine, terbutaline, hexoprenaline, magnesium sulfate, indomethacine and nifedipine or similar agents. Tocolytics suppress muscular contractions of the uterus. Tocolytics are traditionally administered by intravenous, oral or rectal routes. They have suboptimal efficacy and cause significant side effects. In some embodiments, delivery of smaller concentrations of tocolytics directly to the cervix and uterus via the subject device can be more effective with less systemic side effects.

In some embodiments, the device is capable of delivery of an antioxytocic medicament. Pitocin released physiologically by the pituitary gland stimulates uterine contractions. Induction and augmentation of labor via oxytocin intravenous infusion is a standard obstetrical practice. In some embodiments, antibodies to oxytocin or to oxytocin receptors are delivered directly to the cervix via the subject device to effectively inhibit labor.

In some embodiments, the inclusion of a suitable diluent or carrier material to stabilize and maintain the correct concentration of the medicament.

### Biocompatible Intra-Cervical Stent

In some embodiments is provided a medical device, including: (a) at least one biocompatible intra-cervical stent, including a first surface and a second surface; wherein the at least one biocompatible intra-cervical stent can optionally include at least one medicament.

In some embodiments, the at least one biocompatible intra-cervical stent, the at least one biocompatible conduit, or both, can include and may or may not be biodegradable or bioerodable: polylactic acid (PLA), poly-lactic-co-glycolic acid (PLGA), polyglycolic acid (PGA), polyethalene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons, polyanhydrides, polyorthoesters, polyphosphazones, thermoplastic elastomers, polyether block amide (PEBA), fluoropolymers, silicone, latex, animal epithelia, spider or cocoon silk, nano-materials, poly(ethylene-covinyl acetate), hydrophilic polyether urethane (HPEU), polyether urethane (PEU), osmotic agents (e.g. NaCl or sodium acetate), polyhydroalkanoates (PHAs), such as polyhdroxyhexanoate (PHH), polybutylene succinate (PBS), polycaprolactone (PCL), cellulose esters, GD polyvinyl alcohol, or a combination thereof.

Additional material include but are not limited to all FDA-approved and Generally Recognized As Safe (GRAS) materials, including but not limited to EDTA, or non-immunogenic or immunogenic collagens such as but not limited to collagens such as bovine collagen, either alone or in combination with other materials.

In some embodiments, the at least one biocompatible cervical barrier, the at least one biocompatible conduit, or both, include one or more biodegradable materials.

In some embodiments, the at least one biocompatible intra-cervical stent includes substantially a conical shape, optionally once deployed intra-cervically.

In some embodiments, the at least one biocompatible intracervical stent includes substantially a cylindrical shape, optionally once deployed intra-cervically.

In some embodiments, the at least one biocompatible intracervical stent is at least in part hollow.

In some embodiments, the at least one biocompatible intracervical stent includes a first open orifice.

In some embodiments, the first open orifice faces the vagina vhen placed within a subject.

In some embodiments, the at least one biocompatible intracervical stent includes a second open orifice.

In some embodiments, the second open orifice faces the uterus when placed within a subject.

In some embodiments, the at least one biocompatible intra-cervical stem is at least in part elastic.

In some embodiments, the at least one biocompatible intracervical stent is at least in part resilient, and optionally be removable, or optionally be biodegradable over time.

In some embodiments, the at least one biocompatible intra-cervical stent is at least in part collapsible, and optionally delivered intra-cervically via a catheter or catheter-like device.

In some embodiments, the at least one biocompatible intra-cervical stent is at least in part expandable upon proper positioning in the cervix via a catheter or catheter-like device, which positioning optionally can be assisted by, for example, a sonographic or hysteroscopic device.

In some embodiments, the first surface is designed to face the cervical epithelium when placed within a subject.

In some embodiments, the first surface is the outer surface.

In some embodiments, the second surface is designed to face away from the cervical epithelium when placed within a subject.

In some embodiments, the second surface is the inner surface.

In some embodiments, the second surface includes a mesh scaffold or lining, including but not limited to, internal filaments, pilli, nano-materials, or hair-like materials, or fiber-like materials such as any GRAS materials approved for use in the vaginal and cervical compartments such as cocoon silk or cotton or other natural fibers, or inert manmade I synthetic fibers such as nylon, rayon, etc., to trap mucus internally generated in the base and more inward regions of the cervix.

In some embodiments, the medicament includes a therapeutically effective amount of therapeutic agents including: antibiotics, antifungals, antivirals, anti-parasitics, chemotherapies, hormonal therapies, defined selective bacterio-therapy compositions, vaccines, activated charcoal, metal-based compounds, histamines and anti-histamines, immuno-interactive and immunomodulatory agents, hydrogels comparable to the pregnant cervical mucus plug, agents to stimulate restoration of the normal cervical mucus plug in pregnancy, biogels with innate antibacterial properties, tocolytics, anti-oxytotics, pharmaceutically acceptable and safe-for-human-use diluents or carriers, or a combination thereof.

The medicaments can be applied to a reservoir, either open or sealed, in the first or second surface of the device, or both, or impregnated within its biocompatible or biodegradable material, and may include one or more medicaments of the same class or different classes.

The technology for enhanced delivery of medicaments to the vagina and cervical mucosa has been documented in the literature (Cu, Yen, and W. Mark Saltzman. “Controlled surface modification with poly (ethylene) glycol enhances diffusion of PLGA nanoparticles in human cervical mucus.” Molecular Pharmaceutics 6.1 (2008): 173-181; Ensign, Laura M., et al. “Mucus penetrating nanoparticles: biophysical tool and method of drug and gene delivery.” Advanced Materials 24.28 (2012): 3887-3894; Navath, Raghavendra S., et al. “Injectable PAMAM dendrimer—PEG hydrogels for the treatment of genital infections: formulation and in vitro and in vivo evaluation.” Molecular Pharmaceutics 8.4 (2011): 1209-1223). These advances in delivery modalities appear to be ideally suited to use with subject device.

The cervical and vaginal mucosa also present an attractive site for delivery of vaccines to induce active immunity. Kuo-Haller at al reported on favorable IgG and IgA induction in mice via the vaginal rout compared to other routes (Kuo-Haller. Patricia, et al. “Vaccine delivery by polymeric vehicles in the mouse reproductive tract induces sustained local and systemic immunity.” Molecular Pharmaceutics 7.5 (2010): 1585-1595) In some embodiments, the use of vaccines introduced by subject devices induce antibodies against aberrant components of the immune cascade.

In some embodiments, medicaments are delivered by the device, including selective I-kB (IKK) inhibitors to interfere with local cytokine production in order to prevent or treat preterm labor (De Silva, D., M. D. Mitchell, and J. A. Keelan. “Inhibition of choriodecidual cytokine production and inflammatory gene expression by selective I-kB kinase (IKK) inhibitors.” British Journal of Pharmacology 160.7 (2010): 1808-1822).

In some embodiments is provided delivery by subject device of appropriately designed synthetic small interfering RNA to influence targeted gene DNA expression of cytokines or other elements of the immune cascade involved in preterm labor. Recent papers have described many ways that such siRNMs can be protected for delivery to cells and delivered vaginally (Singha, Kaushik, Ran Namgung, and Won Jong Kim. “Polymers in small-interfering RNA delivery.” Nucleic Acid Therapeutics 21.3 (2011): 133-147; Woodrow, Kim A., et al. “Intravaginal gene silencing using biodegradable polymer nanoparticles densely loaded with small-interfering RNA.” Nature Materials 8.6 (2009): 526-533). In some embodiments, the antimicrobials include a therapeutically effective amount of pregnancy-safe therapeutic antibiotics including: amoxicillin, clindamycin, erythromycin, metronidazole, penicillin, gentamicin, ampicillin-sulbactam, cefoxitin, cefotetan, cefazolin, ciprofloxacin, rifampin, vancomycin, azithromycin, ampicilllin-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazoletrimethoprim, or a combination thereof.

Systemically applied antibiotics are often ineffective at treating vaginal bacterial infections in the non-pregnant state or ascending infections in pregnancy which reach the uterine decidua or placental amnion and chorion, where they initiate the immune cascade which triggers preterm labor. Clinical trials using both oral and intravaginal antibiotics to prevent preterm birth have been disappointing (McDonald, Helen Margaret, et al.. “Antibiotics for treating bacterial vaginosis in pregnancy.” The Cochrane Database of Systematic Reviews 1.1 (2007)). In some embodiments, the subject device more effectively delivers antibiotics directly to the vaginal and cervical mucosa by direct proximity and slow release of the antibiotic or antibiotics.

In some embodiments, the antifungals include a therapeutically effective amount of antifungal agents including: imidazole, triazole, thiazole, or similar agents or a combination thereof. Vaginal candida infections are commonly treated with antifungal suppositories and creams. The effectiveness of these modes of delivery is compromised by the tendency of the medication to drain out of the vagina. In some embodiments, the subject device of the disclosure can create a slow, sustained release of antifungals directly into the area of infection which will improve the cure rate and reduce the recurrence rate of these fungal infections.

In some embodiments, the antivirals include a therapeutically effective amount of agents such as but not limited to acyclovir, tenofovir, interferon, imiquimod, or similar agents.

Slow, sustained local cervical-vaginal release of therapeutic levels of these agents from the subject device can be efficacious to treat viruses such as but not limited to herpes simplex virus, HIV, and human papilloma virus. The delivery of imiquimod to treat cervical condyloma and HPV-related cervical dysplasia is preferable.

In some embodiments, the antimicrobials include a therapeutically effective amount of therapeutic anti-parasitic agents including antinematodes, anticestodes, antitrematodes, antiamoebics, antiprotozoals, or similar agents, or a combination thereof. The vagina can provide a reservoir for water-born parasites that is resistant to oral anti-parasitic therapies. The subject device can provide localized treatment.

In some embodiments, the chemotherapies include a therapeutically effective amount of chemotherapies including but not limited to: cisplatin, carboplatin, paclitaxel, topotecan, gemcitabine, docetaxel, ifosfamide, 5-fluorouracil (5-FU), irinotecan, mitomycin, or a combination thereof. Cervical cancer is preceded by progressive degrees of cervical dysplasia. Traditional treatments for dysplasia involve surgical removal of part of the cervix, which may lead to incompetent cervix, late abortion and preterm birth. The sustained local chemotherapy delivery by the subject device and methods to treat cervical dysplasia and thereby prevent cancer and preserve fertility is an attractive application of the present disclosure.

In some embodiments, the hormonal therapies include a therapeutically effective amount of hormonal therapies including but not limited to: estrogen and derivatives thereof, progesterone and derivatives thereof, aldosterone and derivatives thereof, steroids and derivatives thereof, or a combination thereof. Vaginally applied estrogen cream and eluting ring (e.g. Estring™) are currently used to treat cervical-vaginal atrophy. It is reported that “sex hormones and their duration of exposure affect the composition and stability of the microbiome as well as systemic and mucosal immune response” (Brotman, Rebecca M., et al. “Microbiome, sex hormones, and immune responses in the reproductive tract: challenges for vaccine development against sexually transmitted infections.” Vaccine 32.14 (2014): 1543-1552). The present disclosure envisions that co-therapy with hormones by the subject device can improve the efficacy of the other medicaments.

Progesterone delivery by the subject device and methods of the present disclosure can improve efficacy of vaginal progesterone therapy. Progesterone therapy has a documented role in reducing the risk of preterm birth in certain categories of patients. Treatment is by intramuscular 17alpha-hydroxyprogesterone caproate or vaginal natural progesterone gel or capsules. The efficacy of current treatment is suboptimal. (Committee on Practice Bulletins—Obstetrics Practice Bulletin Number 130: Predication and Prevention of Preterm Birth. Washington: The American College of Obstetricians and Gynecologists. Obstetrics and Gynecology, 2012. Print). In some embodiments, improved sustained local delivery of hormones to the cervix and uterus by the subject device, possibly utilizing customized hydrogels or nanotechnology, improves the success of this treatment.

In some embodiments, the defined selective bacteria-therapy compositions include a therapeutically effective amount of beneficial bacteria, including lactic acid-producing or other bacteria. The healthy vaginal bacterial microbiome is known to be dominated by lactobacillus species and lesser numbers of a diverse group of anaerobic and facultative bacteria (Linhares, Tara M., et al. “The vaginal microbiome: New findings bring new opportunities.” Drug Development Research 74.6 (2013): 360-364). Imbalances in this microbiome can lead to pathological conditions, such as preterm delivery (Ganu, Radhika S., Jun Ma, and Kjersti M. Aagaard. “The role of microbial communities in parturition: is there evidence of association with preterm birth and perinatal morbidity and mortality?” American Journal of Perinatology 30.08 (2013): 613-624; Hyman, Richard W., et al. “Diversity of the vaginal microbiome correlates with preterm birth.” Reproductive Sciences 21.1 (2013): 32-40) or post-menopausal vulvovaginal atrophy (Brotman, Rebecca M,, et al, “Association between the vaginal microbiota, menopause status and signs of vulvovaginal atrophy.” Menopause 21.5 (2014): 450-458). One example of the disclosure would be to use the subject device to provide a reservoir of beneficial bacteria to promote a healthy vaginal microbiome in order to prevent or treat preterm birth, vulvovaginal atrophy, or other diseases or conditions.

In some embodiments, vaccination therapy includes vaccines, such as but not limited to, those against influenza, TdaP, Varicella, HPV, Zoster, MMR, Pneumococcal, Meningococcal, Hepatitis A, Hepatitis B.

Recent research has demonstrated that favorable active immunization can be achieved via the vaginal route compared to other routes. Therefore, an additional application for existing vaccines would be via the subject cervical barrier device, possibly including specialized gels or nanotechnology preparations to increase absorption of the vaccines.

In some embodiments, the medicament includes activated charcoal. Activated charcoal has a strong negative electrical charge along with very fine pores which trap bacteria and toxins, preventing the ascent of bacteria up the endocervical canal and absorbing bacterial endotoxins or other harmful metabolites.

In some embodiments, the medicament includes metal based compounds, such as copper-based compounds, silver-based compounds, nickel-based compounds, zinc-based compounds, or a combination thereof. These metal-based compounds have been shown to be safe in human use in such devices as the copper intrauterine contraceptive device, Essure™ contraceptive implants, and specialized dressings for severe skin burns. These metals have antibiotic activity and may prove efficacious in preventing pathological bacteria from ascending the cervical canal, perhaps in combination with other medicaments.

In some embodiments, the medicament includes titanium dioxide, as an anti-microbial and optionally a pigment.

In some embodiments, the medicament includes histamine and antihistamine therapies. Since activation of the immune cascade is tied to preterm labor, one embodiment of the present disclosure includes the case in which local cervical-vaginal histamine/antihistamine therapy delivered by the subject cervical barrier device may help prevent preterm labor by interference with or regulation of one or more elements of the immune system.

In some embodiments, the medicament includes immuno-interactive and immunomodulatory agents, especially functional angonists or antagonists to specific cytokines, chemokines, immunocompetent cells, or other elements of the immune system, including antibodies, binding fragments of antibodies, aptamers, antigens, epitopes, peptide nucleic acids, molecular imprinted biopolymers or chemical polymers, oligonucleotides, antisense oligonucleotides, oligodinucleotides, proteins, peptides, peptidomimetics, nucleic acids, carbohydrates, lipids, small molecule compounds, glucocorticoids, cytostatics, anti-metabolites, anti-CD2 antibody or related binding fragment, anti-CD20 antibody, triple helix molecules, antisense-DNA, anti-sense-RNA, ribozyme, iRNA, miRNA, siRNA, shRNA, cyclosporin, tacrolimus, sirolimus, FTY720, small molecule nuclear factor-kappa B inhibitors, ante-sense compounds, peptide nucleic acids (PNAs) or locked nucleic acids (LNAs), or similar chemical or biological agents. Since it is generally accepted that preterm labor is stimulated by pathological perturbations of the immune inflammatory system, the present disclosure includes the case that the subject device can deliver immune-interactive or immunomodulatory agents directly to the cervix, uterus, placental and fetal compartments for modulation of the abnormal process.

In some embodiments, the medicament can include a hydrogel which mimics the naturally occurring pregnancy cervical mucus plug. The mucus plug can deteriorate prematurely in pregnancy and lead to ascending bacterial infection from the vagina. In some embodiments, artificial mucous is manufactured and held in place inside the cervix by the subject device (for a study of cervical mucus, see Critchfield, Agatha S., et al. “Cervical mucus properties stratify risk for preterm birth.” PLOS ONE 8.8 (2013): e69528; Becher, Naja, et al. “The cervical mucus plug: structured review of the literature.” Acta Obstetricia et Gynecologica Scandinavica 88.5 (2009): 502-513).

In some embodiments, the medicament can include an agent to stimulate the restoration of the normal cervical mucus in pregnancy. In some embodiments, the subject device could be used to deliver appropriate agonist and antagonist agents to stimulate production of the proper mucus from the endocervical glands.

In some embodiments is provided delivery of a specialized biogel with innate antibiotic properties, such as the antibacterial activity exhibited by the amine terminated dendrimer released from a degrading specialized gel (Navath, Raghavendra S., et al. “Injectable PAMAM dendrimer—PEG hydrogels for the treatment of genital infections: formulation and in vitro and in vivo evaluation.” Molecular Pharmaceutics 8.4 (2011): 1209-1223).

In some embodiments is provided delivery of tocolytic agents, such as ritodrine, terbutaline, hexoprenaline, magnesium sulfate, indomethacine and nifedipine or similar agents. Tocolytics suppress muscular contractions of the uterus. Tocolytics are traditionally administered by intravenous, oral or rectal routes. They have suboptimal efficacy and cause significant side effects In some embodiments, delivery of smaller concentrations of tocolytics directly to the cervix and uterus via the subject device can be more effective with less systemic side effects.

In some embodiments is provided delivery via subject device of an antioxytocic medicament. Pitocin released physiologically by the pituitary gland stimulates uterine contractions. Induction and augmentation of labor via oxytocin intravenous infusion is a standard obstetrical practice. In some embodiments antibodies to oxytocin or to oxytocin receptors are delivered directly to the cervix via the subject device to effectively inhibit labor.

In some embodiments, inclusion of a suitable diluent or carrier material to stabilize and maintain the correct concentration of the medicament.

### Biocompatible Vaginal Ring

In some embodiments is provided a medical device, including: at least one biocompatible vaginal ring, wherein the at least one biocompatible vaginal ring can optionally include at least one medicament. The vaginal ring may be included among cervical barriers with all of the functions and features of a cervical barrier as described, previously for the mechanical and physiological protection of the cervix and the cervical mucus plug and for delivery of a full range of medicaments.

In some embodiments is provided a method for treating a subject mammal in need for at least one disease, disorder or condition, including: providing a medical device of the present disclosure including a cervical barrier; and operably engaging the medical device with the subject mammal in need; wherein the subject mammal in need is treated for at least one disease, disorder or condition; and wherein the conduit if necessary permits substantially one-way flow-through of matter.

As used herein, a subject mammal in need may be a human primate, a non-human primate, a research animal, a test animal, an animal bred for racing or hunting, a veterinary animal, a food-source animal, an agricultural animal, a companion animal, an exotic animal, a human patient, or non-human patient.

In some embodiments, a device as described herein adheres to the outer cervical surface of the subject mammal.

In some embodiments, a device as described herein encircles the cervix of the subject mammal

In some embodiments, a device as described herein protects the integrity of the cervical mucus plug of the subject mammal in need.

In some embodiments, a device as described herein prevents invasion of microbes through the cervical opening of the subject mammal in need, preferably by providing a biophysical or mechanical barrier to immigration of bacteria up the cervix, with or without combination with medicaments.

In some embodiments, a device as described herein prevents infection of at least one biological tissues of the subject mammal in need.

As used herein, the at least one biological tissue includes: cervix and cervical tissues, vagina and vaginal epithelium, endometrium, uterine myometrial and decidual layers, placental, amniotic and chorionic compartments, fetal tissues and the fetus itself whose immune system will also react to bacterial invasion or bacterial toxin contamination of the placental compartment, or any combination thereof.

In some embodiments, the treating of a subject mammal in need protects against preterm labor (PTL), preterm birth (PTB), other pregnancy complications, or a combination thereof of the subject mammal in need.

In some embodiments, the treating of a subject mammal in need includes administration of therapeutics for at least one disease, disorder or condition in the subject mammal in need or its fetus.

In some embodiments, the at least one disease, disorder or condition includes: cervical cancer, hormonal imbalances, fungal infections, bacterial vaginosis, chronic inflammatory diseases, or a combination thereof.

In some embodiments, the treating a subject mammal in need includes, but is not limited to: impregnation of medicament within the medical device coating the medicament onto the medical device; application of the medicament by subject mammal in need, or a medical or veterinarian practitioner, in, on, or around the medical device; distribution of the medicament into the vaginal compartment via the conduit of the medical device promoted by dripping or disbursement of medicament through the conduit due to intra-cervical pressure; or a combination thereof.

In some embodiments, the treating a subject mammal in need includes having at least one receptacle designed to house and deliver at least one part of the medicament.

In some embodiments, the treating a subject mammal in need includes pulsatile release of medicament from an electronically controlled reservoir within the medical device.

In some embodiments, the treating a subject mammal in need includes concomitant or simultaneous administration of beneficial selective bacterio-therapies or defined pro biotic mixtures, which promote neonatal digestion of lactal/lacteal proteins during nursing.

In some embodiments, the at least one biocompatible conduit includes at least one one-way valve permitting flow through of matter, bodily fluids, or a combination thereof.

In some embodiments, the at least one biocompatible conduit prevents infection of the chorion, amnion, amniotic fluid, or a combination thereof, by preventing the inward invasion by any bacterial, viral or protozoal species resident in the vaginal compartment of the subject mammal in need, or later introduced by a sexual partner.

In some embodiments, the at least one biocompatible conduit prevents Intra Amniotic Infection (IAI), initiation of inflammatory cascades, which lead to preterm birth (PTB), other pregnancy complications, or a combination thereof.

As provided herein, the diseases, disorders or conditions include, but are not limited to: damage to or degradation of the cervical mucus plug, vaginal microbionte dysbiosis, bacterial vaginosis (BV), vaginitis, recurrent spontaneous abortions (RSA), preterm birth, gestational diabetes, pre-eclampsia including hemolysis elevated liver enzymes low platelets (HELLP Syndrome), preterm premature rupture of the membranes (PPROM), premature rupture of the membranes (PROM), antepartum hemorrhage including placental abruption, chorioamnionitis, intrauterine growth restriction, placenta previa, and sequelae of intraamniotic infection such as inflammatory cytokine cascades leading to premature contractions and birth, cervical dysplasia or cancer, bacterial vaginosis (BV), yeast infection, hormonal imbalances, viral infections, inflammation or chronic inflammation, non-immunized status, or a combination thereof.

As used herein, operably engaging the medical device includes: adhesion to the cervical surface by suction pressure; use of non-suture biocompatible adhesive compositions or chemicals; cervical-facing surfaces with high coefficients of friction within the medical device gripping the outer cervix; creating an initial partial vacuum within the cervix to hold the device in situ; inward-facing nano-surfaces that adhere to the cervix, or a combination thereof.

In some embodiments, operably engaging the medical device includes encircling of the medical device around the cervix, preferably fastening onto it as a tight ring approximately 1-2 centimeters from the distal end of the cervix.

Some embodiments provided herein include a method for treating a subject mammal in need or its fetus for at least one disease, disorder or condition, including: providing a medical device of the present disclosure including a cervical plug; and operably engaging the medical device with the subject mammal in need or its fetus; wherein the subject mammal or its fetus in need is treated for at least one disease, disorder or condition.

As used herein, the subject mammal in need is a human primate, a non-human primate, a research animal, a test animal, a veterinary animal, an animal bred for racing or hunting, a food-source animal, an agricultural animal, a companion animal, an exotic animal, a human patient, or a non-human patient,

In some embodiments, the medical device fits into the cervical canal of the subject mammal in need.

As used herein, the treating a subject mammal in need protects against preterm labor (PTL), preterm birth (PTB), other pregnancy complications, or a combination thereof of the subject mammal in need.

In some embodiments, the treating a subject mammal in need includes administration of therapeutics for at least one disease, disorder or condition in the subject mammal in need or its fetus.

In some embodiments, the at least one disease, disorder or condition includes: cervical cancer, hormonal imbalances, fungal infections, bacterial vaginosis, chronic inflammatory diseases, gestational diabetes, preeclampsia, syndromes or afflictions of the fetus, or a combination thereof. In some embodiments, the treating a subject mammal in need and/or its fetus includes, but is not limited to: impregnation of medicament within the medical device; coating the medicament onto the medical device; application of the medicament by subject mammal in need, or a medical practitioner, in, on, or around the medical device; distribution of the medicament via the conduit of the medical device promoted by dripping of medicament through the conduit due to pressure; or a combination thereof.

In some embodiments, the treating a subject mammal in need includes having at least one receptacle designed to house and deliver at least one part of the medicament.

In some embodiments, the treating a subject mammal in need includes pulsatile release of medicament from an electronically controlled reservoir within the medical device.

In some embodiments, the treating a subject mammal in need includes administration of beneficial selective bacteria-therapies or defined probiotic mixtures, which promote neonatal digestion of lactal/lacteal proteins during nursing, assuming the baby passes through the birth canal.

In some embodiments, the at least one biocompatible conduit includes at least one one-way valve permitting flow through of matter, bodily fluids, or a combination thereof.

In some embodiments, the at least one biocompatible conduit prevents infection of the chorion, amnion, amniotic fluid, or a combination thereof, by preventing the inward invasion by some or any bacterial, viral or protozoal species resident in the vaginal compartment of the subject mammal in need, or later introduced by a sexual partner.

In some embodiments, the at least one biocompatible, preferably one-way or uni-directional conduit, preferably directed towards the vaginal exit, prevents Intra Amniotic Infection (IAI), initiation of inflammatory cascades that lead to preterm birth (PTB), other pregnancy complications, or a combination thereof.

As used herein, the diseases, disorders or conditions include, but are not limited to: damage to or degradation of the cervical mucus plug, vaginal microbiome dysbiosis, bacterial vaginosis (BV), vaginitis, recurrent spontaneous abortions (RSA), preterm birth, gestational diabetes, pre-eclampsia including hemolysis elevated liver enzymes low platelets (HELLP Syndrome), preterm premature rupture of the membranes (PPROM), premature rupture of the membranes (PROM), antepartum hemorrhage including placental abruption, chorioamnionitis, intrauterine growth restriction, placenta previa, and sequelae of intraamniotic infection such as inflammatory cytokine cascades leading to premature contractions and birth, cervical dysplasia or cancer, bacterial vaginosis (BV), yeast infection, hormonal imbalances, viral infections, inflammation or chronic inflammation, non-immunized status, or a combination thereof.

In some embodiments, operably engaging the medical device includes: Velcro™-like or Velcro™ (also sometimes known as “loop and hook” fasteners) outer surface of the medical device gripping the interior surface of the cervical canal; a barbed or abrasive or adhesive surface on the external surface of the medical device, or any other surface with a high coefficient of friction, or any combination thereof.

In some embodiments is provided a method for treating a subject mammal and/or its fetus, preferably via transfer of a therapeutic agent via the chorion, in need for at least one disease, disorder or condition, including: providing a medical device of the present disclosure including a cervical stent; and operably engaging the medical device with the subject mammal in need; wherein the subject mammal in need is treated for at least one disease, disorder or condition.

Some embodiments include the use of a catheter for treating a subject mammal in need for at least one disease, disorder or condition, including: providing a medical device of the present disclosure including a catheter for use throughout the reproductive tract and operably engaging the catheter with the subject mammal in need, wherein the subject mammal in need is treated for at least one disease, disorder or condition.

In some embodiments, the subject mammal in need is a human primate, a non-human primate, a research animal, a test animal, a veterinary animal, an animal bred for racing or hunting, a food-source animal, an agricultural animal, a companion animal, an exotic animal, a patient, or a non-human patient.

In some embodiments, the medical device fits into the cervical canal of subject mammal in need.

In some embodiments, the treating of a subject mammal in need or its fetus includes administration of therapeutics for at least one disease, disorder or condition in the subject mammal in need.

In some embodiments, the treating a subject mammal in need or its fetus includes, but is not limited to: impregnation of medicament within the medical device; coating the medicament onto the medical device; application of the medicament by subject mammal in need, or a medical practitioner, in, on, or around the medical device; distribution of the medicament via the conduit of the medical device promoted by dripping of medicament through the stent due to pressure; or a combination thereof.

In some embodiments, the treating a subject mammal in need or its fetus includes having at least one receptacle designed to house and deliver at least one part of the medicament.

In some embodiments, the treating of a subject mammal in need or its fetus includes pulsatile release of medicament from an electronically-controlled reservoir within the medical device.

In some embodiments, the treating a subject mammal in need includes administration of beneficial selective bacterio-therapies or defined pro biotic mixtures, which promote later neonatal digestion of lactal/lacteal proteins during nursing, assuming the child passes through the birth canal, or if born C-section, assuming the mother's vaginal microbiome is transferred to the neonate via a vaginal swab of the mother, which is then transferred by any means to the child including but not limited to, oral or rectal insertion.

In some embodiments, the diseases, disorders or conditions include, but are not limited to: damage to or degradation of the cervical mucus plug, vaginal microbiome dysbiosis, bacterial vaginosis (BV), vaginitis, recurrent spontaneous abortions (RSA), preterm birth, gestational diabetes, pre-eclampsia including hemolysis elevated liver enzymes low platelets (HELLP Syndrome), preterm premature rupture of the membranes (PPROM), premature rupture of the membranes (PROM), antepartum hemorrhage including placental abruption, chorioamnionitis, intrauterine growth restriction, placenta previa, and sequelae of intraamniotic infection such as inflammatory cytokine cascades leading to premature contractions and birth, cervical dysplasia or cancer, bacterial vaginosis (BV), yeast infection, hormonal imbalances, viral infections, inflammation or chronic inflammation, non-immunized status, or a combination thereof.

In some embodiment, operably engaging the medical device is done by inserting the intra-cervical stent within the cervix of a subject mammal in need including, but not limited to, either via catheter, sonogram-guided insertion, hysteroscopically directed insertion, or manually by a qualified medical or veterinarian professional via speculum.

In some embodiments, the intra-cervical stent functions as a scaffold or matrix that promotes the regrowth of the cervical mucus plug.

In some embodiments, the intra-cervical stent degrades while the cervical mucus plug forms or regenerates.

In some embodiments, the intra-cervical stent degrades at a rate that takes at least 1 week.

In some embodiments, the intra-cervical stent degrades at a rate that takes at least 4 weeks, or a duration determined appropriate by a qualified medical or veterinarian professional.

In some embodiments is provided a method for treating a subject mammal in need for at least one disease, disorder or condition, including: providing a medical device including a vaginal ring; and operably engaging the medical device with the subject mammal in need; wherein the subject mammal in need is treated for at least one disease, disorder or condition.

### EXAMPLES

### Example 1

### Cervical Barrier

A cervical barrier made of appropriate material, such as but not limited to silicone, such as is generally known in the art as a birth control diaphragm is made, or a vaginal pessary to treat vaginal, uterine, rectal or urinary bladder prolapse is made, or a drug delivery ring to deliver hormonal contraceptives or estrogen therapy is made.

The cervical barrier can include a medicament as is described herein, either integral to the material, applied upon the surface thereof, contained within a reservoir, or a combination thereof as is known in the art.

A hole is produced in the barrier using methods known in the art, such as by puncture or melting, in a configuration that can accept a conduit of the present disclosure, such as but not limited to a valve. The hole is placed anywhere in the barrier material, including, but not limited to, hypoallergenic latex as appropriate, but preferably central and to be localized at or near the cervical os when placed within a subject.

A conduit of the present disclosure, if required, made of an appropriate material such as but not limited to silicone, is provided and attached to the barrier such as to allow flow of matter from one side of the barrier to the other through the hole in the barrier when in operation. The barrier and the conduit can be made of the same or different material. The barrier and conduit can be attached to each other using appropriate methods and materials, such as but not limited to melting, adhesives, sonic or heat welding, and the like.

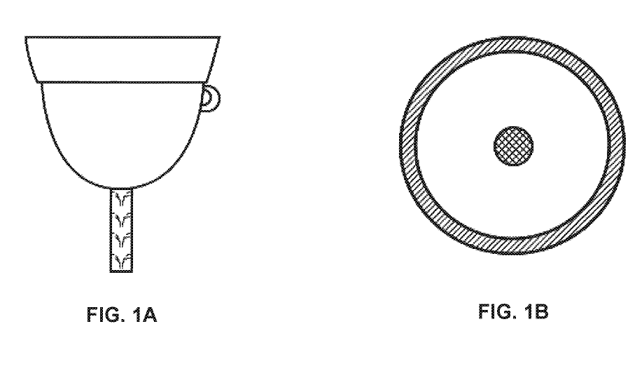

One embodiment of the present disclosure is depicted generally in FIG. 1. The device is made using the general materials and methods described herein and as are known in the art or later developed.

### Example 2

### Cervical Plug

A cervical plug made of appropriate material, such as but not limited to silicone, such as is generally known in the art as a pelvic floor support pessary is made as is known in the art.

The cervical plug is designed to be inserted into the cervical canal, and thus will be smaller than a pelvic floor support pessary which is meant to be placed in the vagina.

The cervical plug may incorporate a structure to help hold it in the cervix, which may include among other configurations spikes or brushes that project from the outer surface or an inflatable balloon at the apex.

The cervical plug may be composed of a material which swells contact with moisture or cervical fluids, thus holding the plug in place.

The cervical plug may be composed of biodegradable materials which obviate the need to remove the plug after its use is no longer required.

The cervical plug can include a medicament as is described herein, either integral to the material, applied upon the surface thereof, contained in a reservoir, or a combination thereof as is known in the art.

The cervical plug may include a conduit if necessary, within its internal diameter such as but not limited to a valve. The conduit is configured to allow flow of matter unidirectionally or bidirectionally from one side of the cervical plug to the other when in operation.

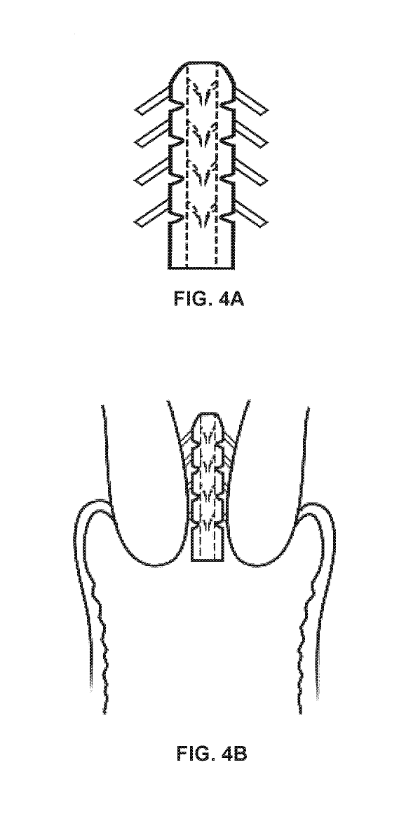

One embodiment of the present disclosure is depicted generally in FIG. 4. The device is made using the general materials and methods described herein and as are known in the art or later developed.

### Example 3

### Cervical Stent

A cervical stent made of appropriate material, usually semi-rigid such as is generally known in the art as a cardiac artery stent is made. The stent begins as a collapsed structure. After the clinician places the stent in the cervical canal, an internal balloon is inflated, which expands the stent to the maximum diameter permitted by the walls of the cervical canal. The expansion of the stent under pressure stretches the cervix open and holds the stent in place.

The cervical stent may be employed to treat cervical stenosis. Cervical stenosis may be the cause of infertility, hematometria, and endometriosis. In this case, the surgeon would surgically dilate the cervix under at least partial anesthesia prior to placement of the stent. The stent would remain in place until the cervix had healed, thus preventing re-scarring and reclosure of the cervical canal. Delivery of antibiotics or anti-inflammatory agents by the stent may be included in the treatment. The patent cervical canal allows sperm to pass through the cervix and menstrual fluid to drain properly.

The cervical stent may be used to treat infertility secondary to inadequate cervical mucus which presents a barrier to sperm penetration. The stent may be deployed into the cervical canal to deliver antibiotics to treat the underlying infection of the endocervical glands and histamines, estrogen or other agents to stimulate the glands to produce the thin, shiny stretchable ovulatory mucus that is necessary to facilitate sperm passage. This treatment would be accomplished shortly prior to either spontaneous or medically induced ovulation.

The cervical stent may be used to restore the cervical mucus plug during pregnancy. The expanded stent with an internal architecture could provide a scaffold on which the cervical mucus plug would form and accumulate. Alternatively, the core of the stent could provide a reservoir for an artificial hydrogel used to replace the natural cervical mucus plug and possibly containing antimicrobial agents, including antibodies. In this instance, the stent may have a plug which could be inserted in the vaginal hollow end to keep the hydrogel in place and the plug may have an injectable port which would allow the clinician to replenish the hydrogel without the need to replace the stent.

The endocervical stent could also be used to induce cervical ripening prior to induction of labor. Current clinical practice to stimulate the cervix to soften and dilate include mechanical (placement of a Foley catheter with fully inflated balloon up inside the cervix) or medical (placement of a prostaglandin in the vagina (dinoprostone as Cervidil® or PGF2alpha provided by a compounding pharmacy). Neither practice is optimally effective. The cervical stent could be dilated within the cervical canal and used to deliver prostaglandins or other agents directly to the cervix and the placental membranes.

The cervical stent could be used to deliver medicament to the placental membranes and the fetus. The dilated stent would provide space to hold a preparation containing agents such as antibodies, selective I-kB kinase (IKK) inhibitors, or small blocking RNA to modulate abnormal amounts or species of proteins or nucleotides causing harm to the fetus. The preparation may be composed of nanoparticles such as PLGA which protect the medicament from enzymatic degradation in bodily fluids or tissues (Lee, Jeong Yu, et al. “Prolonged gene silencing by siRNA/chitosan-g-deoxycholic acid polyplexes loaded within biodegradable polymer nanoparticles.” Journal of Controlled Release 162.2 (2012): 407-413).

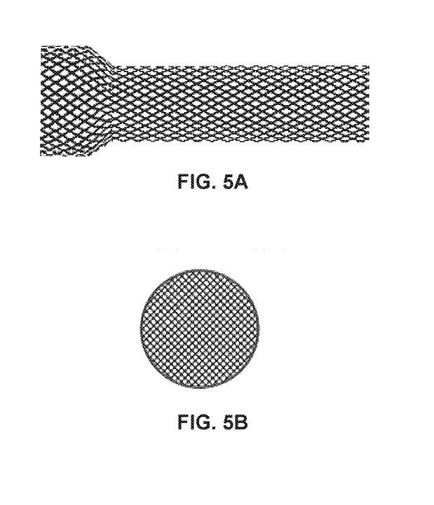

The cervical stent may include a constructed internal conduit, such as but not limited to a valve. The hollow core of the stent is also ipso facto its own a two way conduit. The conduit is configured to allow flow of matter either unidirectionally or bidirectionally from one side of the cervical plug to the other when in operation. One embodiment of the present disclosure is depicted generally in FIG. 5. The device is made using the general materials and methods described herein and as are known in the art or later developed.

### Example 4

### Cervical Ring

A cervical ring made of appropriate material, such as but not limited to silicone, such as is generally known in the art as a drug delivery ring to deliver hormonal contraceptives or estrogen therapy is made.

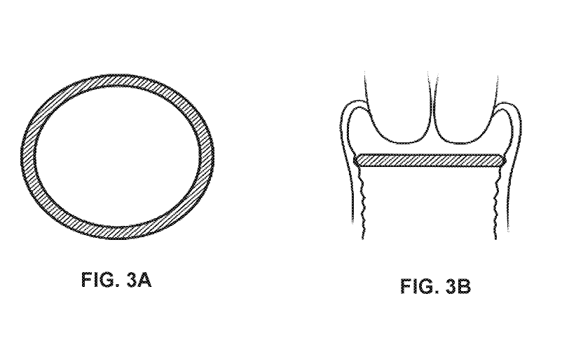

The cervical ring include a medicament as is described herein, either integral to the material, applied upon the surface thereof, contained within a reservoir, or a combination thereof as is known in the art. One embodiment of the present disclosure is depicted generally in FIG. 3. The device is made using the general materials and methods described herein and as are known in the art or later developed.

### Example 5

### Method of Using a Cervical Barrier

A subject in need for treatment of short cervix in the second trimester of pregnancy is fitted with a cervical barrier of the present disclosure having the following characteristics: strong structure, firmly seated in the upper vagina, completely covering the cervix, deploying a reservoir pre-filled with clindamycin to destroy pathological bacteria and composed of an impregnated material such as histamine which releases gradually to stimulate the production of cervical mucus.

The device is provided with a conduit and a one-way valve which prevents the excessive build-up of secretions above the device. The device is composed of materials which biodegrade over a set period of time. The clinician may select a device with a degradation time suitable for the subject and circumstances at hand. At the completion of the treatment interval, the clinician may perform a vaginal ultrasound to assess the state of the cervical plug. The device may be replaced with a fresh device as needed, either by the clinician or the patient, and the treatment is repeated until the cervical mucus plug has been restored or the patient has reached a safe point in pregnancy for delivery.

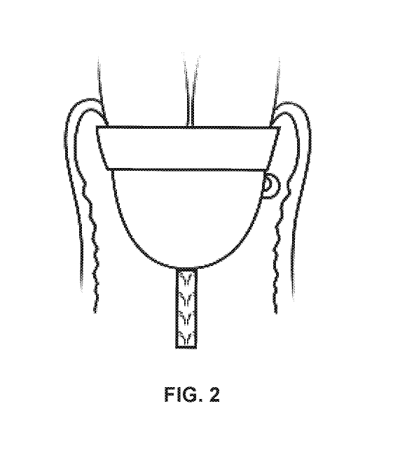

One embodiment of the present disclosure is depicted generally in FIG. 2, showing the device engaged with a subject. The device is made using the general materials and methods described herein and as are known in the art or later developed.

### Example 6

### Method of Using a Cervical Plug

A subject pregnant mammal in need of treatment has a depleted cervical mucus plug during mid-pregnancy. The cervix is short and has already dilated 1 cm. It may be too late to apply the cervical barrier for protection. Instead, a cervical plug is gently inserted into the open cervix by the clinician. The length and diameter of the plug are elected to meet the subject mammal and the circumstances at hand. The plug absorbs water slightly after insertion; the small amount of swelling anchors the plug in place. The center of the plug contains a biogel with very fine pores which resist the passage of bacteria from the vagina. The inner surface of the plug elutes clindamycin, metronidazole and selected antibodies against common pathological vaginal bacteria to eliminate the ascent of these bacteria, and an agent which stimulates secretion of cervical mucus. The cervical plug biodegrades over several weeks. At that time, the clinician performs a vaginal ultrasound in the office setting to examine the cervical plug. If the mucus plug is restored, the treatment is completed and the patient is followed closely. If the mucus plug has not been satisfactorily restored, a new plug is inserted. One embodiment of the present disclosure is depicted generally in FIG. 4(B), showing the device engaged with a subject. The device is made using the general materials and methods described herein and as are known in the art or later developed.

### Example 7

### Method of Using a Cervical Stent

A 30 year old women who has not yet had children presents with grade 3 cervical dysplasia and a positive test for high risk HPV virus infection. The physician performs a colposcopy and determines that the abnormal area extends up inside the endocervical canal. Normally, this would require a surgical procedure to remove the abnormal area, which might compromise future fertility. The physician places a cervical stent into the cervical canal and expands the stent using the associated balloon. The procedure is accomplished under topical lidocaine. The stent elutes a therapeutic, continuous dose of imiquimod directly into the cervical canal over several weeks, which destroys the virus and the abnormal tissue. The stent is composed of a biodegradable material and does not require removal. This stent does not require a conduit, since the open channel of the stent permits adequate drainage and retrograde flow of vaginal fluids is not contraindicated. The cervix naturally closes back down over several days after the stent completely degrades.

One embodiment of the present disclosure is depicted generally in FIG. 5(B), showing the device engaged with a subject. The device is made using the general materials and methods described herein and as are known in the art or later developed.

### Example 8

### Method of Using a Cervical Ring

A subject in need for treatment of short cervix in the second trimester of pregnancy is fitted with a cervical ring of the present disclosure having the following characteristics: strong structure, firmly seated in the upper vagina, completely covering the cervix, deploying a reservoir pre-filled with clindamycin to destroy pathological bacteria and composed of an impregnated material such as histamine which releases gradually to stimulate the production of cervical mucus.

The clinician may select a device with a degradation time suitable for the subject and circumstances at hand. At the completion of the treatment interval, the clinician may perform a vaginal ultrasound to assess the state of the cervical plug. The device may be replaced with a fresh device as needed, either by the clinician or the patient, and the treatment is repeated until the cervical mucus plug has been restored or the patient has reached a safe point in pregnancy for delivery.

One embodiment of the present disclosure is depicted generally in FIG. 2, showing the device engaged with a subject. The device is made using the general materials and methods described herein and as are known in the art or later developed.

All headings are for the convenience of the reader and should not be used to limit the meaning of the text that follows the heading, unless so specified.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

### Embodiments

While various aspects and embodiments have been disclosed herein, other aspects and embodiments will be apparent to those skilled in the art. Various example embodiments of the disclosure can be described according to the following embodiments.

In some embodiments is provided a medical device, including at least one biocompatible cervical barrier, including a first surface and a second surface and at least one biocompatible conduit. In some embodiments, the at least one cervical barrier and the at least one conduit are operably linked to allow passage of matter through the medical device. In some embodiments, the at least one biocompatible cervical barrier, the at least one biocompatible conduit, or a combination thereof, includes at least one medicament,

In some embodiments, the at least one biocompatible cervical barrier, the at least one biocompatible conduit, or both, include polylactic acid (PLA), poly(lactic-co-glycolic acid) (PLGA), polyglycolic acid (PGA), polyethylene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons or silk derivatives, polyanhydrides, polyorthoesters, polyphosphazones, thermoplastic elastomers, including but not limited to polyether block amide (PEBA), fluoropolymers, silicone, latex or hypoallergenic latex, all materials listed on FDA-approved Generally Recognizes As Safe (GRAS) list, ethylenediaminetetraacetic acid (EDTA), animal epithelia or non-immunogenic collagens, nano-materials, or a combination thereof.

In some embodiments, the at least one biocompatible cervical barrier, the at least one biocompatible conduit, or both, include one or more biodegradable materials. In some embodiments, the at least one biocompatible cervical barrier, the at least one biocompatible conduit, or both, include one or more non-biodegradable materials.

In some embodiments, the at least one biocompatible cervical barrier is substantially dome shaped. In some embodiments, the at least one biocompatible cervical barrier is substantially flat shaped. In some embodiments, the at least one biocompatible cervical barrier includes at least one rim. In some embodiments, the at least one biocompatible cervical barrier is at least in part elastic. In some embodiments, the at least one biocompatible cervical barrier is at least in part resilient.

In some embodiments, the first surface is designed to face away from the cervix when placed within a subject. In some embodiments, the first surface is substantially convex. In some embodiments, the first surface has a protrusion housing the conduit. In some embodiments, the second surface is designed to face toward the cervix when placed within a subject mammal. In some embodiments, the second surface is substantially concave. In some embodiments, the second surface has a perforation leading to the conduit.

In some embodiments, the at least one biocompatible conduit includes at least one one-way valve facing away from the cervix when placed within a subject. In some embodiments, the at least one one-way valve is a bicuspid valve, a multicuspid valve, a globe valve, a ball valve, a needle valve, a pinch valve, a plug valve, a relief valve, or a check valve.

In some embodiments, the at least one biocompatible conduit permits passage of matter through the medical device. In some embodiments, the at least one biocompatible cervical barrier and the at least one biocompatible conduit are operably linked at the apex of the medical device. In some embodiments, the at least one biocompatible cervical barrier, and the at least one biocompatible conduit are operably linked within the protrusion. In some embodiments, the at least one biocompatible cervical barrier, and the at least one biocompatible conduit are operably linked within the perforation on the second surface.

In some embodiments, the medicament includes a therapeutically effective amount of a therapeutic agent, including, for example antimicrobials, chemotherapies, hormonal therapies, antivirals, selective bacterio-therapy compositions, or a combination thereof.

In some embodiments, the antimicrobials include a therapeutically effective amount of pregnancy safe therapeutic antibiotics, including, for example, amoxicillin, ampicillin, clindamycin, erythromycin, metronidazole, penicillin, gentamicin, ampicillin, sulbactam, cefoxitin, cefotetan, cefazolin, ciprofloxacin, penicillin G, penicillin V. rifampin, vancomycin, azithromycin, ampicillin-sulbactam, ampicillian-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazole I trimethoprim, or a combination thereof.

In some embodiments, the antimicrobials include a therapeutically effective amount of antifungal agents, including, for example imidazole, triazole, thiazole, or a combination thereof.

In some embodiments, the antimicrobials includes a therapeutically effective amount of therapeutic anti-parasitic agents, including, for example, antinematodes, anticestodes, antitrematodes, anti amoebics, antiprotozoals, or a combination thereof.

In some embodiments, the chemotherapies include a therapeutically effective amount of cisplatin, carboplatin, paclitaxel, topotecan, gemcitahine, docetaxel, ifosfamide, 5-fluorouracil (5-FU), irinotecan, mitomycin, or a combination thereof.

In some embodiments, the hormonal therapies include a therapeutically effective amount of estrogen and derivatives thereof, progesterone and derivatives thereof, aldosterone and derivatives thereof, steroids and derivatives thereof, or a combination thereof.

In some embodiments, the antivirals include a therapeutically effective amount of vaccines including influenza vaccinations.

In some embodiments, the selective bacteria-therapy compositions include a therapeutically effective amount of beneficial aerobic bacteria, including, for example, lactic-acid bacteria.

In some embodiments, the medicament includes activated charcoal, copper based compounds, silver based compounds, nickel-based compounds, zinc based compounds, or titanium dioxide.

In some embodiments, the medicament includes histamine and antihistamine therapies.

In some embodiments, the medicament includes aptamers, peptide nucleic acids, molecular imprinted biopolymers, chemical polymers. In some embodiments, the medicament includes a single or a group of drugs or an anti-ligand, including but not limited to antibiotics, chemo, metals, aptarners, peptide nucleic acids and/or molecular imprinted biopolymers.

In some embodiments is provided a method of using the cervical barrier as described herein. In some embodiments, the method includes treating a subject mammal in need for at least one disease, disorder or condition. In some embodiments, the method includes providing a cervical barrier medical device and operably engaging the medical device with the subject mammal in need; wherein the subject mammal in need is treated for at least one disease, disorder or condition. In some embodiments, the conduit permits substantially one-way flow through of matter.

In some embodiments, the subject mammal in need is a human primate, a non-human primate, a research animal, a test animal, a veterinary animal, a food-source animal, an agricultural animal, a companion animal, an exotic animal, a human patient, or non-human patient.

In some embodiments, the cervical barrier adheres to the cervical surface of the subject mammal. In some embodiments, the cervical barrier encircles the cervix of the subject mammal. In some embodiments, the cervical barrier protects the integrity of the cervical mucus plug of the subject mammal in need. In some embodiments, the cervical barrier prevents invasion of microbes through the cervical opening of the subject mammal in need. In some embodiments, the cervical barrier prevents infection of at least one biological tissues of the subject mammal in need.

In some embodiments, the at least one biological tissue includes cervix and cervical tissues, vagina and vaginal epithelium, endometrium, uterine myometrial and decidual layers, placental, amniotic and chorionic compartments, or a combination thereof

In some embodiments, treating a subject mammal in need protects against preterm labor (PTL), preterm birth (PTB), other pregnancy complications, or a combination thereof of the subject mammal in need.

In some embodiments, treating a subject mammal in need includes administration of therapeutics for at least one disease, disorder or condition in the subject mammal in need.

In some embodiments, the at least one disease, disorder or condition includes, for example, cervical cancer, hormonal imbalances, fungal infections, bacterial vaginosis, chronic inflammatory diseases, or a combination thereof.

In some embodiments, treating a subject mammal in need includes, but is not limited to, impregnation of medicament within the cervical barrier, coating the medicament onto the cervical barrier, application of the medicament by subject mammal in need, or a medical practitioner, in, on, or around the cervical barrier, distribution of the medicament via the conduit promoted by dripping of medicament through the conduit due to pressure, or a combination thereof.

In some embodiments, treating a subject mammal in need includes having at least one receptacle designed to house and deliver at least one part of the medicament.

In some embodiments, treating a subject mammal in need includes pulsatile release of medicament from an electronically controlled reservoir within the medical device. In some embodiments, treating a subject mammal in need includes administration of beneficial selective bacteria-therapies or defined pro biotic mixtures, which promote neonatal digestion of lactal/lacteal proteins during nursing. In some embodiments, the method results in regrowth of the cervical mucus plug of the subject mammal in need.

In some embodiments, the at least one biocompatible conduit includes at least one one-way valve permitting flow through of matter, bodily fluids, or a combination thereof.

In some embodiments, at least one biocompatible conduit prevents infection of the chorion, amnion, amniotic fluid, or a combination thereof, by preventing the inward invasion by any bacterial species of the anatomies.

In some embodiments, the at least one biocompatible conduit prevents Intra Amniotic Infection (IAI), initiation of inflammatory cascades, which lead to preterm birth (PTB), other pregnancy complications, or a combination thereof.

In some embodiments, the disease, disorder or condition include, but are not limited to, preterm labor, preterm birth, intraamniotic infection, cervical cancer, bacterial Vaginosis, yeast infection, hormonal imbalances, viral infections, inflammation or chronic inflammation, or a combination thereof.

In some embodiments, operably engaging the cervical barrier includes adhesion to the cervical surface by suction pressure, use of non-suture biocompatible adhesive compositions or chemicals, velcro-like surfaces within the medical device gripping the outer cervix, vacuum-assisted wound-closure mechanisms, inward facing nano-surfaces that adhere to the cervix, or a combination thereof. In some embodiments, operably engaging the cervical barrier includes encircling of the cervical barrier around the cervix, thereby fastening the cervical barrier to the cervix.

In some embodiments, is provided a medical device, wherein the medical device includes at least one biocompatible intra-cervical plug. In some embodiments, the intra-cervical plug includes a first surface and a second surface. In some embodiments, the medical device includes at least one biocompatible conduit. In some embodiments, the at least biocompatible one intra-cervical plug and the at least one conduit are operably linked to allow passage of matter through the medical device. In some embodiments, the at least one biocompatible intra-cervical plug, the at least one biocompatible conduit, or a combination thereof, include at least one medicament

In some embodiments, the at least one biocompatible cervical barrier, the at least one biocompatible conduit, or both, include polylactic acid (PLA), poly(lactic-co-glycolic acid) (PLGA), polyglycolic acid (PGA), polyethylene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons or silk derivatives, polyanhydrides, polyorthoesters, polyphosphazones, thermoplastic elastomers, including but not limited to polyether block amide (PERM, fluoropolymers, silicone, latex or hypoallergenic latex, all materials listed on FDA-approved Generally Recognizes As Safe (GRAS) list, ethylenediaminetetraacetic acid (EDTA), animal epithelia or non-immunogenic collagens, nano-materials, or a combination thereof.

In some embodiments, the at least one biocompatible intra-cervical plug, at least one biocompatible conduit, or both, include one or more biodegradable materials. In some embodiments, the at least one biocompatible intra-cervical plug, at least one biocompatible conduit, or both, include one or more non-biodegradable materials.

In some embodiments, the at least one biocompatible intra-cervical plug is substantially conical shaped. In some embodiments, the at least one biocompatible intra-cervical plug is substantially cylindrical shaped. In some embodiments, the at least one biocompatible intra-cervical plug is substantially hollow. In some embodiments, the at least one biocompatible intra-cervical plug includes a first open orifice. In some embodiments, the first open orifice faces the vagina when placed within a subject. In some embodiments, the at least one biocompatible intra-cervical plug includes a second open orifice. In some embodiments, the second open orifice faces the utenis when placed within a subject In some embodiments, the at least one biocompatible intra-cervical plug is at least in part elastic in some embodiments, the at least one biocompatible intra-cervical plug is at least in part resilient. In some embodiments, the first surface is designed to face the cervical epithelium when placed within a subject. In some embodiments, the first surface includes barbs, prongs, spikes, thistle, bristles, quills, spurs, a biofilm, or combination thereof. In some embodiments, the first surface is the outer surface.In some embodiments, the second surface is designed to face away from the cervical epithelium when placed within a subject mammal. In some embodiments, the second surface is the inner surface. In some embodiments, the second surface houses the at least one biocompatible conduit,

In some embodiments, the at least one biocompatible conduit includes at least one one-way valve facing the first open orifice. In some embodiments, the at least one one-way valve is a bicuspid, a multicuspid, a globe, a ball, a needle, a pinch, a plug, a relief, or a check valve.

In some embodiments, the at least one biocompatible conduit permits passage of matter through the medical device. In some embodiments, the at least one biocompatible intra-cervical plug and the at least one biocompatible conduit are operably linked at the second surface. In some embodiments, the at least one biocompatible intra-cervical plug and the at least one biocompatible conduit are operably linked and are positioned at the first open orifice, the second open orifice, or an intermediary position thereof.

In some embodiments, the medicament includes a therapeutically effective amount of a therapeutic agent, including, for example antimicrobials, chemotherapies, hormonal therapies, antivirals, selective bacterio-therapy compositions, or a combination thereof.

In some embodiments, the antimicrobials include a therapeutically effective amount of pregnancy safe therapeutic antibiotics, including, for example, amoxicillin, ampicillin, clindamycin, erythromycin, metronidazole, penicillin, gentamicin, ampicillin, sulbactam, cefoxitin, cefotetan, cefazolin, ciprofloxacin, penicillin G, penicillin V, rifampin, vancomycin, azithromycin, ampicillin-sulbactam, ampicillian-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazole I trimethoprim, or a combination thereof.

In some embodiments, the antimicrobials include a therapeutically effective amount of antifungal agents, including, for example imidazole, triazole, thiazole, or a combination thereof.

In some embodiments, the antimicrobials includes a therapeutically effective amount of therapeutic anti-parasitic agents, including, for example, antinematodes, anticestodes, antitrematodes, antiamoebics, antiprotozoals, or a combination thereof.

In some embodiments, the chemotherapies include a therapeutically effective amount of cisplatin, carboplatin, paclitaxel, topotecan, gemcitabine, docetaxel, ifosfamide, 5-fluorouracil (5-FU), irinotecan, mitomycin, or a combination thereof.

In some embodiments, the hormonal therapies include a therapeutically effective amount of estrogen and derivatives thereof, progesterone and derivatives thereof, aldosterone and derivatives thereof, steroids and derivatives thereof, or a combination thereof.

In some embodiments, the anti virals include a therapeutically effective amount of vaccines including influenza vaccinations.

In some embodiments, the selective bacteria-therapy compositions include a therapeutically effective amount of beneficial aerobic bacteria, including, for example, lactic-acid bacteria.

In some embodiments, the medicament includes activated charcoal, copper based compounds, silver based compounds, nickel-based compounds, zinc based compounds, or titanium dioxide.

In some embodiments, the medicament includes histamine and antihistamine therapies.

In some embodiments, the medicament includes aptamers, peptide nucleic acids, molecular imprinted biopolymers, chemical polymers. In some embodiments, the medicament includes a single or a group of drugs or an anti-ligand, including but not limited to antibiotics, chemo, metals, aptamers, peptide nucleic acids and/or molecular imprinted biopolymers.

In some embodiments is provided a method of using the intra-cervical plug as described herein. In some embodiments, the method includes treating a subject mammal in need for at least one disease, disorder or condition. In some embodiments, the method includes providing the intra-cervical plug and operably engaging the intra-cervical plug with the subject mammal in need. In some embodiments, the subject mammal in need is treated for at least one disease, disorder or condition.

In some embodiments, the subject mammal in need is a human primate, a non-human primate, a research animal, a test animal, a veterinary animal, a food-source animal, an agricultural animal, a companion animal, an exotic animal, a human patient, or a non-human patient.

In some embodiments, the intra-cervical plug fits into the cervical canal of the subject mammal in need.

In some embodiments, treating a subject mammal in need protects against preterm labor (PTL), preterm birth (PTB), other pregnancy complications, or a combination thereof of the subject mammal in need.

In some embodiments, treating a subject mammal in need includes administration of therapeutics for at least one disease, disorder or condition in the subject mammal in need.

In some embodiments, the at least one disease, disorder or condition includes cervical cancer, hormonal imbalances, fungal infections, bacterial vaginosis, chronic inflammatory diseases, or a combination thereof.

In some embodiments, treating a subject mammal in need includes, but is not limited to, impregnation of medicament within the intra-cervical plug, coating the medicament onto the intra-cervical plug, application of the medicament by subject mammal in need, or a medical practitioner, in, on, or around the intra-cervical plug, distribution of the medicament via the conduit, promoted by dripping of medicament through the conduit due to pressure, or a combination thereof.

In some embodiments, treating a subject mammal in d includes having at least one receptacle designed to house and deliver at least one part of the medicament. In some embodiments, treating a subject mammal in need includes pulsatile release of medicament from an electronically controlled reservoir within the medical device. In some embodiments, treating a subject mammal in need includes administration of beneficial selective bacterio-therapies or defined probiotic mixtures, which promote neonatal digestion of lactal/lacteal proteins during nursing. In some embodiments, the method results in regrowth of the cervical mucus plug of the subject mammal in need.

In some embodiments, the at least one biocompatible conduit includes at least one one-way valve permitting flow through of matter, bodily fluids, or a combination thereof. In some embodiments, at least one biocompatible conduit prevents infection of the chorion, amnion, amniotic fluid, or a combination thereof, by preventing the inward invasion by any bacterial species of the anatomies. In some embodiments, the at least one biocompatible conduit prevents Intra Amniotic Infection (IAT), initiation of inflammatory cascades, which lead to preterm birth (PTB), other pregnancy complications, or a combination thereof. In some embodiments, the disease, disorder or condition include, but are not limited to: preterm labor, preterm birth, intraamniotic infection, cervical cancer, bacterial vaginosis, yeast infection, hormonal imbalances, viral infections, inflammation or chronic inflammation, or a combination thereof.

In some embodiments, operably engaging the intra-cervical plug includes gripping of the cervical canal by Velcro-like outer surface of the medical device or barbed surface on the external surface of the medical device, or a combination thereof.

In some embodiments is provided a medical device including at least one biocompatible intra-cervical stent. In some embodiments, the intra-cervical stent includes a first surface and a second surface. In some embodiments, the at least one biocompatible intra-cervical stent includes at least one medicament.

In some embodiments, the at least one biocompatible intra-cervical stent, the at least one biocompatible conduit, or both, include polylactic acid (PLA), poly(lactic-co-glycolic acid) (PLGA), polyglycolic acid (PGA), polyethylene glycol (PEG), polycaprolactone (PCL), polydioxanone (PDO), poly (lactide-co-glycolide), polyglyconate, polyhydroxybutyrate (PHB), polyhydroxyvalerate (PHV), poly-pseudo amino acids, polyesteramides, silk cocoons or silk derivatives, polyanhydrides, polyorthoesters, polyphosphazones, thermoplastic elastomers, including but not limited to polyether block amide (PEBA), fluoropolymers, silicone, latex or hypoallergenic latex, all materials listed on FDA-approved Generally Recognizes As Safe (GRAS) list, ethylenediaminetetraacetic acid (EDTA), animal epithelia or non-immunogenic collagens, nano-materials, or a combination thereof.

In some embodiments, the at least one biocompatible intra-cervical stent, the at least one biocompatible conduit, or both, include one or more biodegradable materials. In some embodiments, the at least one biocompatible intra-cervical stent, the at least one biocompatible conduit, or both, include one or more nonbiodegradable materials.

In some embodiments, the at least one biocompatible intra-cervical stent is substantially conical shaped. In some embodiments, the at least one biocompatible intra-cervical stent is substantially cylindrical shaped. In some embodiments, the at least one biocompatible intra-cervical stent is at least in part hollow. In some embodiments, the at least one biocompatible intra-cervical stent includes a first open orifice. In some embodiments, the first open orifice faces the vagina when placed within a subject. In some embodiments, the at least one biocompatible intra-cervical stent includes a second open orifice. In some embodiments, the second open orifice faces the uterus when placed within a subject. In some embodiments, the at least one biocomptible intra-cervical stent is at least in part elastic. In some embodiments, the at least one biocompatible intra-cervical stent is at least in part resilient. In some embodiments, the at least one biocompatible intra-cervical stent is at least in part collapsible. In some embodiments, the at least one biocompatible intra-cervical stent is at least in part expandable. In some embodiments, the first surface is designed to face the cervical epithelium when placed within a subject. In some embodiments, the first surface is the outer surface. In some embodiments, the second surface is designed to face away from the cervical epithelium when placed within a subject mammal. In some embodiments, the second surface is the inner surface. In some embodiments, the second surface includes a mesh scaffold.

In some embodiments, the medicament includes a therapeutically effective amount of a therapeutic agent, including, for example antimicrobials, chemotherapies, hormonal therapies, antivirals, selective bacterio-therapy compositions, or a combination thereof.

In some embodiments, the antimicrobials include a therapeutically effective amount of pregnancy sate therapeutic antibiotics, including, for example, amoxicillin, ampicillin, clindamycin, erythromycin, metronidazole, penicillin, gentamicin, ampicilllin, sulbactam, cefoxitin, cefotetan, cefazolin, ciprofloxacin, penicillin G, penicillin V, rifampin, vancomycin, azithromycin, ampicillin-sulbactam, ampicillian-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazole I trimethoprim, or a combination thereof.

In some embodiments, the antimicrobials include a therapeutically effective amount of antifungal agents, including, for example imidazole, triazole, thiazole, or a combination thereof.

In some embodiments, the antimicrobials includes a therapeutically effective amount of therapeutic anti-parasitic agents, including, for example, antinematodes, anticestodes, antitrematodes, antiamoebics, antiprotozoals, or a combination thereof.

In some embodiments, the chemotherapies include a therapeutically effective amount of cisplatin, carboplatin, paclitaxel, topotecan, gemcitabine, docetaxel, ifosfamide, 5-fluorouracil (5-FU), irinotecan, mitomycin, or a combination thereof.

In some embodiments, the hormonal therapies include a therapeutically effective amount of estrogen and derivatives thereof, progesterone and derivatives thereof, aldosterone and derivatives thereof, steroids and derivatives thereof, or a combination thereof.

In some embodiments, the antivirals include a therapeutically effective amount of vaccines including influenza vaccinations.

In some embodiments, the selective bacteria-therapy compositions include a therapeutically effective amount of beneficial aerobic bacteria, including, for example, lactic-acid bacteria.

In some embodiments, the medicament includes activated charcoal, copper based compounds, silver based compounds, nickel-based compounds, zinc based compounds, or titanium dioxide.

In some embodiments, the medicament includes histamine and antihistamine therapies.

In some embodiments, the medicament includes aptamers, peptide nucleic acids, molecular imprinted biopolymers, chemical polymers, in some embodiments, the medicament includes a single or a group of drugs or an anti-ligand, including but not limited to antibiotics, chemo, metals, aptamers, peptide nucleic acids and/or molecular imprinted biopolymers.

In some embodiments is provided a method of using the intra-cervical stent. In some embodiments, the method includes treating a subject mammal in need for at least one disease, disorder or condition, including providing the intra-cervical stent, and operably engaging the intra-cervical stent with the subject mammal in need. In some embodiments, the subject mammal in need is treated for at least one disease, disorder or condition, in some embodiments, the intra-cervical stent fits into the cervical canal of the subject mammal in need.

In some embodiments, the subject mammal in need is a human primate, a non-human primate, a research animal, a test animal, a veterinary animal, a food-source animal, an agricultural animal, a companion animal, an exotic animal, a patient, or a nonhuman patient.

In some embodiments, treating a subject mammal in need protects against preterm labor (PTL), preterm birth (PTB), other pregnancy complications, or a combination thereof of the subject mammal in need. In some embodiments, treating a subject mammal in need includes administration of therapeutics for at least one disease, disorder or condition in the subject mammal in need.

In some embodiments, the at least one disease, disorder or condition includes: cervical cancer, hormonal imbalances, fungal infections, Bacterial Vaginosis, chronic inflammatory diseases, or a combination thereof.

In some embodiments, treating a subject mammal in need includes, but is not limited to impregnation of medicament within the intra-cervical stent, coating the medicament onto the intra-cervical stent, application of the medicament by subject mammal in need, or a medical practitioner, in, on, or around the intra-cervical stent, distribution of the medicament via the conduit, promoted by dripping of medicament through the conduit due to pressure, or a combination thereof.

In some embodiments, treating a subject mammal in need includes having at least one receptacle designed to house and deliver at least one part of the medicament. In some embodiments, treating a subject mammal in need includes pulsatile release of medicament from an electronically controlled reservoir within the medical device. In some embodiments, treating a subject mammal in need includes administration of beneficial selective bacterio-therapies or defined probiotic mixtures, which promote neonatal digestion of lactal/lacteal proteins during nursing.

In some embodiments, the method results in regrowth of the cervical mucus plug of the subject mammal in need.

In some embodiments, the disease, disorder or condition include, but are not limited to preterm labor, preterm birth, intraamniotic infection, cervical cancer, bacterial vaginosis, yeast infection, hormonal imbalances, viral infections, inflammation or chronic inflammation, or a combination thereof.

In some embodiments, operably engaging the intra-cervical stent is done by inserting the intra-cervical stent within the cervix of a subject mammal in need.

In some embodiments, the intra-cervical stent functions as a scaffold that promotes the regrowth of the cervical mucus plug. In some embodiments, the intra-cervical stent degrades while the cervical mucus plug forms. In some embodiments, the intra-cervical stent degrades at a rate that takes at least 1 week. In some embodiments, the intra-cervical stent degrades at a rate that takes at least 4 weeks.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

What is claimed is:

1. A medical device, comprising:
a biocompatible cervical implant; and
a biocompatible conduit;
wherein the biocompatible cervical implant and the biocompatible conduit are operably linked to allow passage of matter through the medical device, and
wherein the biocompatible cervical implant or the biocompatible conduit comprise at least one medicament.

2. The medical device of claim 1, wherein the biocompatible cervical implant is a cervical barrier, a cervical plug, a cervical stent, or a cervical ring.

3. The medical device of claim 1, wherein the biocompatible cervical implant or the biocompatible conduit comprises polylactic acid (PLA), poly(lactic-co-glycolic acid) (PLGA), polyglycolic acid (PGA), polyethylene glycol (PEG), polycaprolactone (PCL), polydioxanone (PDO), poly(lactide-co-glycolide), polyglyconate, polyhydroxybutyrate (PHB), polyhydroxyvalerate (PHV), poly-pseudo amino acids, polyesteramides, silk cocoons or silk derivatives, polyanhydrides, polyorthoesters, polyphosphazones, thermoplastic elastomers, polyether block amide (PEBA), fluoropolymers, silicone, latex or hypoallergenic latex, all materials listed on FDA-approved Generally Recognizes As Safe (GRAS) list, ethylenediaminetetraacetic acid (EDTA), animal epithelia or non-immunogenic collagens, nano-materials, or a combination thereof.

4. The medical device of claim 1, wherein the biocompatible cervical implant or the biocompatible conduit comprises one or more biodegradable materials.

5. The medical device of claim 1, wherein the biocompatible cervical implant or the biocompatible conduit comprises one or more non-biodegradable materials.

6. The medical device of claim 1, wherein the biocompatible conduit comprises at least one one-way valve facing away from the cervix when placed within a subject.

7. The medical device of claim 6, wherein the at least one one-way valve is a bicuspid valve, a multicuspid valve, a globe valve, a ball valve, a needle valve, a pinch valve, a relief valve, or a check valve.

8. The medical device of claim 1, wherein the matter is a bodily fluid, secretion, bacteria, toxin, or foreign matter.

9. The medical device of claim 1, wherein the medicament comprises a therapeutically effective amount of a therapeutic agent selected from an antimicrobial, a chemotherapy, a hormonal therapy, an antiviral, a selective bacterio-therapy composition, or a combination thereof.

10. The medical device of claim 9, wherein the antimicrobial comprises a therapeutically effective amount of amoxicillin, ampicillin, clindamycin, erythromycin, metronidazole, penicillin, gentamicin, ampicillin, sulbactam, cefoxitin, cefotetan, cefazolin, ciprofloxacin, penicillin G, penicillin V, rifampin, vancomycin, azithromycin, ampicillin-sulbactam, ampicillan-clavulanic acid, cephalosporins, levofloxacin, quinolones, chloramphenicol, nitrofurantoin, cefuroxime, cefaclor, cefadroxil, cefamandole, cefpodoxime, cefprozil, ceftazidime, ceftibuten, ceftizoxime, ceftriaxone, cephalexin, cephapirin, cephradine, cefdinir, cefditoren, cefepime, cefixime, sulfamethoxazole I trimethoprim, imidazole, triazole, thiazole, antinematodes, anticestodes, antitrematodes, antiamoebics, antiprotozoals, or a combination thereof.

11. The medical device of claim 9, wherein the chemotherapy comprises a therapeutically effective amount of cisplatin, carboplatin, paclitaxel, topotecan, gemcitabine, docetaxel, ifosfamide, 5-fluorouracil (5-FU), irinotecan, mitomycin, or a combination thereof.

12. The medical device of claim 9, wherein the hormonal therapy comprises a therapeutically effective amount of estrogen and derivatives thereof, progesterone and derivatives thereof, aldosterone and derivatives thereof, steroids and derivatives thereof, or a combination thereof.

13. The medical device of claim 9, wherein the antiviral comprises a therapeutically effective amount of vaccines comprising influenza vaccinations.

14. The medical device of claim 9, wherein the selective bactedo-therapy composition is a therapeutically effective amount of beneficial aerobic bacteria or a lactic-acid bacteria.

15. The medical device of claim 1, wherein the medicament is one of activated charcoal, copper based compounds, silver based compounds, nickel-based compounds, zinc based compounds, titanium dioxide, or a combination thereof.

16. The medical device of claim 1, wherein the medicament comprises histamine and antihistamine therapies.

17. The medical device of claim 1, wherein the medicament comprises aptamers.

18. The medical device of claim 1, wherein the medicament comprises peptide nucleic acids.

19. The medical device of claim 1, wherein the medicament is one of molecular imprinted biopolymers or chemical polymers.
